(19) United States
(12) Patent Application Publication
(10) Pub. No.: US 2023/0192864 A1
(43) Pub. Date: Jun. 22, 2023

(54) Treatment Of Lung Conditions With Integrin Subunit Alpha 1 (ITGA1) Inhibitors

(71) Applicant: Regeneron Pharmaceuticals, Inc., Tarrytown, NY (US)

(72) Inventors: Manuel Allen Revez Ferreira, Tarrytown, NY (US); Julie E. Horowitz, Tarrytown, NY (US); Katherine Siminovitch, Tarrytown, NY (US); Goncalo Abecasis, Tarrytown, NY (US); Aris Baras, Tarrytown, NY (US); Maria Cristina Suciu, Tarrytown, NY (US)

(21) Appl. No.: 18/067,085

(22) Filed: Dec. 16, 2022

Related U.S. Application Data
(60) Provisional application No. 63/291,194, filed on Dec. 17, 2021.

(51) Int. Cl. C07K 16/28; A61K 31/7105

(52) U.S. Cl. CPC C07K 16/2839; A61K 31/7105

(57) ABSTRACT

The present disclosure provides methods of treating subjects having a lung disease such as a fibrotic lung disease, pulmonary fibrosis, interstitial lung disease, chronic obstructive pulmonary disease (COPD), or asthma, or at risk of developing a lung disease, and methods of identifying subjects having an increased risk of developing a lung disease.

### REFERENCE TO SEQUENCE LISTING

This application includes a Sequence Listing submitted electronically as an XML file named 381203630SEQ, created on Nov. 30, 2022, with a size of 86,876 kilobytes. The Sequence Listing is incorporated herein by reference.

### FIELD

The present disclosure relates generally to the treatment of subjects having a lung disease, such as a fibrotic lung disease, pulmonary fibrosis, interstitial lung disease, chronic obstructive pulmonary disease (COPD), or asthma, or at risk of developing a lung disease, with Integrin Subunit Alpha 1 (ITGA1) inhibitors, and methods of identifying subjects having an increased risk of developing a lung disease.

### BACKGROUND

Fibrotic pulmonary diseases are progressive and irreversible. Standard therapies are mere palliative as they cannot address the underlying disease mechanism once the subject has progressed to a point at which symptoms are present. Thus, there is a long-felt but unmet need in the field for a method of treating asymptomatic subjects as well as those who are at risk of developing fibrotic pulmonary diseases to prevent onset of the disease, delay onset of the disease, or reduce the severity of disease symptoms, The methods of the disclosure provide a preventative or efficacious treatment, as opposed to a merely palliative treatment, for asymptomatic subjects as well as those subjects at risk of developing the disease.

Integrin Subunit Alpha 1 (ITGA1) is a component of cell surface integrin receptor that heterodimerizes with the beta 1 subunit to form a cell-surface receptor for collagen and laminin. It recognizes the proline-hydroxylated sequence G-F-P-G-E-R in collagen, and is involved in anchorage-dependent, negative regulation of EGF-stimulated cell growth. It acts on cell binding to laminin and collagen and neurite outgrowth, and peripheral nerve regeneration. Proper collagen-integrin interaction is important in fracture healing, suggesting that the ITGA1 gene is involved in mesenchymal stem cell proliferation and chondrogenesis. The heterodimeric receptor is also involved in cell-cell adhesion and may play a role in inflammation and fibrosis.

### SUMMARY

The present disclosure provides methods of treating a subject having a fibrotic lung disease or at risk of developing a fibrotic lung disease, the methods comprising administering an ITGA1 inhibitor to the subject.

The present disclosure also provides methods of treating a subject having pulmonary fibrosis or at risk of developing pulmonary fibrosis, the methods comprising administering an ITGA1 inhibitor to the subject.

The present disclosure also provides methods of treating a subject having interstitial lung disease or at risk of developing interstitial lung disease, the methods comprising administering an ITGA1 inhibitor to the subject.

The present disclosure also provides methods of treating a subject having chronic obstructive pulmonary disease (COPD) or at risk of developing COPD, the methods comprising administering an ITGA1 inhibitor to the subject.

The present disclosure also provides methods of treating a subject having asthma or at risk of developing asthma, the methods comprising administering an ITGA1 inhibitor to the subject.

The present disclosure also provides methods of treating a subject with a lung disease therapeutic agent, wherein the subject has a fibrotic lung disease, pulmonary fibrosis, interstitial lung disease, COPD, or asthma, or is at risk of developing a fibrotic lung disease, pulmonary fibrosis, interstitial lung disease, COPD, or asthma, the methods comprising: determining whether the subject has an ITGA1 variant nucleic acid molecule by: obtaining or having obtained a biological sample from the subject; and performing or having performed a sequence analysis on the biological sample to determine if the subject has a genotype comprising the ITGA1 variant nucleic acid molecule; and administering or continuing to administer the lung disease therapeutic agent in a standard dosage amount to a subject that is ITGA1 reference, and/or administering an ITGA1 inhibitor to the subject; and administering or continuing to administer the lung disease therapeutic agent in an amount that is the same as or less than a standard dosage amount to a subject that is heterozygous for the ITGA1 variant nucleic acid molecule, and/or administering an ITGA1 inhibitor to the subject; wherein the presence of a genotype having the ITGA1 variant nucleic acid molecule indicates the subject has a reduced risk of developing the fibrotic lung disease, pulmonary fibrosis, interstitial lung disease, COPD, or asthma.

The present disclosure also provides methods of identifying a subject having an increased risk of developing a fibrotic lung disease, pulmonary fibrosis, interstitial lung disease, COPD, or asthma, the methods comprising: determining or having determined the presence or absence of an ITGA1 variant nucleic acid molecule in a biological sample obtained from the subject; wherein: when the subject is ITGA1 reference, then the subject has an increased risk of developing a fibrotic lung disease, pulmonary fibrosis, interstitial lung disease, COPD, or asthma; and when the subject is heterozygous or homozygous for an ITGA1 variant nucleic acid molecule, then the subject has a decreased risk of developing a fibrotic lung disease, pulmonary fibrosis, interstitial lung disease, COPD, or asthma.

The present disclosure also provides lung disease therapeutic agents for use in the treatment of a lung disease in a subject identified as having an ITGA1 variant nucleic acid molecule, or the complement thereof, wherein the variant nucleic acid molecule has a nucleotide sequence that comprises a nucleotide at a position corresponding to the nucleotide at the variant position of an ITGA1 variant nucleic acid molecule, or the complement thereof.

The present disclosure also provides ITGA1 inhibitors for use in the treatment of a fibrotic lung disease, pulmonary fibrosis, interstitial lung disease, COPD, or asthma in a subject that: a) is reference for an ITGA1 genomic nucleic acid molecule, an ITGA1 mRNA molecule, or an ITGA1 cDNA molecule; or b) is heterozygous for an ITGA1 variant genomic nucleic acid molecule, or the complement thereof; an ITGA1 variant mRNA molecule, or the complement thereof; or an ITGA1 variant cDNA molecule, or the complement thereof.

### BRIEF DESCRIPTION OF THE DRAWINGS

The accompanying figures, which are incorporated in and constitute a part of this specification, illustrate several features of the present disclosure.

FIG. 1 shows association of rare pLoFs <1% (M1.1) in ITGA1 with various immune-related and respiratory-related quantitative traits.

FIG. 2 shows association of rare pLoFs <1% (M1.1) in ITGA1 with various immune-related and respiratory-related diseases. The red bar indicates burden of pLoFs results in better lung function, and a trend for protection against asthma and COPD.

### DESCRIPTION

Various terms relating to aspects of the present disclosure are used throughout the specification and claims. Such terms are to be given their ordinary meaning in the art, unless otherwise indicated. Other specifically defined terms are to be construed in a manner consistent with the definitions provided herein.

Unless otherwise expressly stated, it is in no way intended that any method or aspect set forth herein be construed as requiring that its steps be performed in a specific order. Accordingly, where a method claim does not specifically state in the claims or descriptions that the steps are to be limited to a specific order, it is in no way intended that an order be inferred, in any respect. This holds for any possible non-expressed basis for interpretation, including matters of logic with respect to arrangement of steps or operational flow, plain meaning derived from grammatical organization or punctuation, or the number or type of aspects described in the specification.

As used herein, the singular forms “a,” “an” and “the” include plural referents unless the context clearly dictates otherwise.

As used herein, the term “about” means that the recited numerical value is approximate and small variations would not significantly affect the practice of the disclosed embodiments. Where a numerical value is used, unless indicated otherwise by the context, the term “about” means the numerical value can vary by ±10% and remain within the scope of the disclosed embodiments.

As used herein, the term “comprising” may be replaced with “consisting” or “consisting essentially of” in particular embodiments as desired.

As used herein, the term “isolated”, in regard to a nucleic acid molecule or a polypeptide, means that the nucleic acid molecule or polypeptide is in a condition other than its native environment, such as apart from blood and/or animal tissue. In some embodiments, an isolated nucleic acid molecule or polypeptide is substantially free of other nucleic acid molecules or other polypeptides, particularly other nucleic acid molecules or polypeptides of animal origin. In some embodiments, the nucleic acid molecule or polypeptide can be in a highly purified form, i.e., greater than 95% pure or greater than 99% pure. When used in this context, the term “isolated” does not exclude the presence of the same nucleic acid molecule or polypeptide in alternative physical forms, such as dimers or alternatively phosphorylated or derivatized forms.

As used herein, the terms “nucleic acid”, “nucleic acid molecule”, “nucleic acid sequence”, “polynucleotide”, or “oligonucleotide” can comprise a polymeric form of nucleotides of any length, can comprise DNA and/or RNA, and can be single-stranded, double-stranded, or multiple stranded. One strand of a nucleic acid also refers to its complement.

As used herein, the term “subject” includes any animal, including mammals. Mammals include, but are not limited to, farm animals (such as, for example, horse, cow, pig), companion animals (such as, for example, dog, cat), laboratory animals (such as, for example, mouse, rat, rabbits), and non-human primates (such as, for example, apes and monkeys). In some embodiments, the subject is a human. In some embodiments, the subject is a patient under the care of a physician. In some embodiments, the subject is a smoker. In some embodiments, the subject is a smoker of a tobacco product.

Rare variants in the ITGA1 gene associated with a decreased risk of developing a lung disease in humans has been identified in accordance with the present disclosure. It has been further observed in accordance with the present disclosure that the burden of ITGA1 loss-of-function variant nucleic acid molecules (whether these variations are homozygous or heterozygous in a particular subject) associates with a decreased risk of developing a lung disease. Moreover, the identification by the present disclosure of the association between additional variants and gene burden masks indicates that ITGA1 itself (rather than linkage disequilibrium with variants in another gene) is responsible for a protective effect in lung disease. It is believed that no variants of the ITGA1 gene or protein have any known association with lung disease, such as pulmonary fibrosis, interstitial lung disease, chronic obstructive pulmonary disease, or asthma. Altogether, the genetic analyses described herein surprisingly indicate that the ITGA1 gene and, in particular, loss-of-function variants in the ITGA1 gene, associate with a decreased risk of developing a fibrotic lung disease. Therefore, subjects that are ITGA1 reference that have an increased risk of developing a fibrotic lung disease may be treated such that the fibrotic lung disease is prevented, the symptoms thereof are reduced, and/or development of symptoms is repressed. Accordingly, the present disclosure provides methods of leveraging the identification of such variants in subjects to identify or stratify risk in such subjects of developing a fibrotic lung disease, or to diagnose subjects as having an increased risk of developing a fibrotic lung disease, such that subjects at risk or subjects with active disease may be treated accordingly.

For purposes of the present disclosure, any particular human can be categorized as having one of three ITGA1 genotypes: i) ITGA1 reference; ii) heterozygous for an ITGA1 variant nucleic acid molecule; or iii) homozygous for an ITGA1 variant nucleic acid molecule. A human is ITGA1 reference when the human does not have a copy of an ITGA1 variant nucleic acid molecule. A human is heterozygous for an ITGA1 variant nucleic acid molecule when the human has a single copy of an ITGA1 variant nucleic acid molecule. A human is homozygous for an ITGA1 variant nucleic acid molecule when the human has two copies of an ITGA1 variant nucleic acid molecule. A human who has an ITGA1 polypeptide having a partial loss-of-function (or predicted partial loss-of-function) is hypomorphic for ITGA1.

As used herein, an ITGA1 variant nucleic acid molecule is any ITGA1 nucleic acid molecule (such as, a genomic nucleic acid molecule, an mRNA molecule, or a cDNA molecule) encoding an ITGA1 polypeptide having a partial loss-of-function, a complete loss-of-function, a predicted partial loss-of-function, or a predicted complete loss-of-function. The ITGA1 variant nucleic acid molecule can also be a missense variant, a splice-site variant, a stop-gain variant, a start-loss variant, a stop-loss variant, a frameshift variant, or an in-frame indel variant, or a variant that encodes a truncated ITGA1 predicted loss-of-function polypeptide. An ITGA1 variant nucleic acid molecule can also be any nucleic acid molecule (such as, a genomic nucleic acid molecule, an mRNA molecule, or a cDNA molecule) resulting in complete loss or decreased or aberrant expression of ITGA1 mRNA or polypeptide. An ITGA1 variant nucleic acid molecule can also be any missense variant nucleic acid molecule (such as, a genomic nucleic acid molecule, an mRNA molecule, or a cDNA molecule) resulting in a reduction of ITGA1 activity.

Suitable examples of ITGA1 variant nucleic acid molecules, such as ITGA1 loss-of-function variant nucleic acid molecules, include the following variations (Chromosome:Position(GRCh38.p13):Reference Allele:Alternate Allele): 5:52920430:C:T, 5:52944942:G:T, 5:52910243:G:T, 5:52918899:G:C, 5:52918730:A:T, 5:52952404:CA:C, 5:52861561:T:C, 5:52939693:T:C, 5:52910201:C:T, 5:52788416:T:C, 5:52788356:G:A, 5:52893783:G:T, 5:52920332:AT:A, 5:52952436:GA:G, 5:52939945:G:T, 5:52925371:C:T, 5:52939690:TG:T, 5:52898385:T:C, 5:52897529:GT:G, 5:52937490:C:G, 5:52947412:CT:C, 5:52882022:G:T, 5:52939946:T:A, 5:52861503:A:AT, 5:52881929:T:G, 5:52932134:C:A, 5:52939933:AC:A, 5:52933993:C:G, 5:52927645:TCCTG:T, 5:52939934:CT:C, 5:52893841:G:C, 5:52881939:G:T, 5:52932134:C:G, 5:52864814:GA:G, 5:52849381:C:A, 5:52922818:CT:C, 5:52925286:TG:T, 5:52865083:G:A, 5:52939606:C:CAT, 5:52915509:C:CGTGGTGA, 5:52865083:G:T, 5:52944999:ATC:A, 5:52910194:TA:T, 5:52947462:G:A, 5:52898323:C:T, 5:52945006:GT:G, 5:52910292:CT:C, 5:52861560:G:A, 5:52939642:G:GA, 5:52864979:GC:G, 5:52927590:CA:C, 5:52918814:G:T, 5:52788351:G:GC, 5:52920469:G:A, 5:52905808:A:AT, 5:52922876:GT:G, 5:52861501:C:CT, 5:52865771:T:A, 5:52947456:TGG:T, 5:52918736:C:T, 5:52920376:C:T, 5:52915496:G:GA, 5:52898346:GTC:G, 5:52947462:G:T, 5:52920444:C:A, 5:52882022:G:A, 5:52927640:TC:T, 5:52925470:G:GT, 5:52952430:GA:G, 5:52937516:T:C, 5:52915570:G:GC, 5:52861530:C:A, 5:52932046:G:A, 5:52893841:G:T, 5:52887965:TGTAA:T, 5:52937399:AG:A, 5:52887863:AG:A, 5:52905910:T:A, 5:52925304:TA:T, 5:52910222:C:T, 5:52887906:C:T, 5:52910199:AG:A, 5:52909042:G:A, 5:52898269:TA:T, 5:52939588:A:C, 5:52937515:G:GT, 5:52865745:C:A, 5:52937474:GT:G, 5:52925299:G:T, 5:52920367:CA:C, 5:52898296:CAG:C, 5:52925283:CT:C, 5:52893817:GAGAA:G, 5:52915565:TG:T, 5:52933892:A:T, 5:52915524:G:T, 5:52887846:C:T, 5:52937515:G:A, 5:52929670:GA:G, 5:52915463:G:A, 5:52905900:G:T, 5:52865688:AG:A, 5:52933993:C:A, 5:52932137:G:A, 5:52788355:T:C, 5:52897529:G:A, 5:52947344:G:A, 5:52910417:CAAGT:C, 5:52864772:C:A, 5:52939677:C:T, 5:52918807:C:A, 5:52915502:TTTTGG:T, 5:52893693:C:T, 5:52939855:A:T, 5:52925489:T:C, 5:52939673:C:CA, 5:52887966:G:T, 5:52918877:GA:G, and 5:52947463:T:C. The alternate allele(s) is/are a variant nucleotide(s). The position is a variant position.

For subjects that are genotyped or determined to be ITGA1 reference, such subjects have an increased risk of developing a fibrotic lung disease, pulmonary fibrosis, interstitial lung disease, COPD, or asthma. For subjects that are genotyped or determined to be either ITGA1 reference or heterozygous for an ITGA1 variant nucleic acid molecule, such subjects can be treated with an ITGA1 inhibitor.

In any of the embodiments described throughout the present disclosure, the lung disease is a fibrotic lung disease, pulmonary fibrosis, interstitial lung disease, COPD, or asthma. In any of the embodiments described throughout the present disclosure, the lung disease is a fibrotic lung disease. In any of the embodiments described throughout the present disclosure, the lung disease is a pulmonary fibrosis. In any of the embodiments described throughout the present disclosure, the lung disease is interstitial lung disease. In any of the embodiments described throughout the present disclosure, the lung disease is chronic obstructive pulmonary disease. In any of the embodiments described throughout the present disclosure, the lung disease is asthma.

Symptoms of a lung disease, such as a fibrotic lung disease, include, but are not limited to, shortness of breath (dyspnea)′ a dry cough, fatigue, unexplained weight loss, aching muscles and joints, and widening and rounding of the tips of the fingers or toes (clubbing).

The present disclosure provides methods of treating a subject having a fibrotic lung disease or at risk of developing a fibrotic lung disease, the methods comprising administering an ITGA1 inhibitor to the subject.

The present disclosure also provides methods of treating a subject having pulmonary fibrosis or at risk of developing pulmonary fibrosis, the methods comprising administering an ITGA1 inhibitor to the subject.

The present disclosure also provides methods of treating a subject having interstitial lung disease or at risk of developing interstitial lung disease, the methods comprising administering an ITGA1 inhibitor to the subject.

The present disclosure also provides methods of treating a subject having COPD or at risk of developing COPD, the methods comprising administering an ITGA1 inhibitor to the subject.

The present disclosure also provides methods of treating a subject having asthma or at risk of developing asthma, the methods comprising administering an ITGA1 inhibitor to the subject.

In some embodiments, the ITGA1 inhibitor comprises an inhibitory nucleic acid molecule. In some embodiments, the inhibitory nucleic acid molecule comprises an antisense molecule, a small interfering RNA (siRNA) molecule, or a short hairpin RNA (shRNA) molecule. In some embodiments, the inhibitory nucleic acid molecule comprises an antisense molecule. In some embodiments, the inhibitory nucleic acid molecule comprises an siRNA molecule. In some embodiments, the inhibitory nucleic acid molecule comprises an shRNA molecule. Such inhibitory nucleic acid molecules can be designed to target any region of an ITGA1 nucleic acid molecule, such as an mRNA molecule. In some embodiments, the inhibitory nucleic acid molecule hybridizes to a sequence within an ITGA1 genomic nucleic acid molecule or mRNA molecule and decreases expression of the ITGA1 polypeptide in a cell in the subject. In some embodiments, the ITGA1 inhibitor comprises an antisense molecule that hybridizes to an ITGA1 genomic nucleic acid molecule or mRNA molecule and decreases expression of the ITGA1 polypeptide in a cell in the subject. In some embodiments, the ITGA1 inhibitor comprises an siRNA that hybridizes to an ITGA1 genomic nucleic acid molecule or mRNA molecule and decreases expression of the ITGA1 polypeptide in a cell in the subject. In some embodiments, the ITGA1 inhibitor comprises an shRNA that hybridizes to an ITGA1 genomic nucleic acid molecule or mRNA molecule and decreases expression of the ITGA1 polypeptide in a cell in the subject.

In some embodiments, the antisense nucleic acid molecules comprise or consist of any of the nucleotide sequences represented by SEQ ID NOs: 32-18299. In some embodiments, the siRNA molecules comprise or consist of any of the nucleotide sequences (sense and antisense strands presented one after the other) represented by SEQ ID NOs: 18300-69677 (e.g., the sense strand is, for example, SEQ ID NO: 18300 and the corresponding antisense strand is SEQ ID NO: 18301; the sense strand is, for example, SEQ ID NO: 18302 and the corresponding antisense strand is SEQ ID NO: 18303; etc.).

The inhibitory nucleic acid molecules can comprise RNA, DNA, or both RNA and DNA. The inhibitory nucleic acid molecules can also be linked or fused to a heterologous nucleic acid sequence, such as in a vector, or a heterologous label. For example, the inhibitory nucleic acid molecules can be within a vector or as an exogenous donor sequence comprising the inhibitory nucleic acid molecule and a heterologous nucleic acid sequence. The inhibitory nucleic acid molecules can also be linked or fused to a heterologous label. The label can be directly detectable (such as, for example, fluorophore) or indirectly detectable (such as, for example, hapten, enzyme, or fluorophore quencher). Such labels can be detectable by spectroscopic, photochemical, biochemical, immunochemical, or chemical means. Such labels include, for example, radiolabels, pigments, dyes, chromogens, spin labels, and fluorescent labels. The label can also be, for example, a chemiluminescent substance; a metal-containing substance; or an enzyme, where there occurs an enzyme-dependent secondary generation of signal. The term “label” can also refer to a “tag” or hapten that can bind selectively to a conjugated molecule such that the conjugated molecule, when added subsequently along with a substrate, is used to generate a detectable signal. For example, biotin can be used as a tag along with an avidin or streptavidin conjugate of horseradish peroxidate (HRP) to bind to the tag, and examined using a calorimetric substrate (such as, for example, tetramethylbenzidine (TMB)) or a fluorogenic substrate to detect the presence of HRP. Exemplary labels that can be used as tags to facilitate purification include, but are not limited to, myc, HA, FLAG or 3XFLAG, 6XHis or polyhistidine, glutathione-S-transferase (GST), maltose binding protein, an epitope tag, or the Fc portion of immunoglobulin. Numerous labels include, for example, particles, fluorophores, haptens, enzymes and their calorimetric, fluorogenic and chemiluminescent substrates and other labels.

The inhibitory nucleic acid molecules can comprise, for example, nucleotides or non-natural or modified nucleotides, such as nucleotide analogs or nucleotide substitutes. Such nucleotides include a nucleotide that contains a modified base, sugar, or phosphate group, or that incorporates a non-natural moiety in its structure. Examples of non-natural nucleotides include, but are not limited to, dideoxynucleotides, biotinylated, aminated, deaminated, alkylated, benzylated, and fluorophor-labeled nucleotides.

The inhibitory nucleic acid molecules can also comprise one or more nucleotide analogs or substitutions. A nucleotide analog is a nucleotide which contains a modification to either the base, sugar, or phosphate moieties. Modifications to the base moiety include, but are not limited to, natural and synthetic modifications of A, C, G, and T/U, as well as different purine or pyrimidine bases such as, for example, pseudouridine, uracil-5-yl, hypoxanthin-9-yl (I), and 2-aminoadenin-9-yl. Modified bases include, but are not limited to, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo (such as, for example, 5-bromo), 5-trifluoromethyl and other 5-substituted uracils and cytosines, 7-methylguanine, 7-methyladenine, 8-azaguanine, 8-azaadenine, 7-deazaguanine, 7-deazaadenine, 3-deazaguanine, and 3-deazaadenine.

Nucleotide analogs can also include modifications of the sugar moiety. Modifications to the sugar moiety include, but are not limited to, natural modifications of the ribose and deoxy ribose as well as synthetic modifications. Sugar modifications include, but are not limited to, the following modifications at the 2′ position: OH; F; O—, S—, or N-alkyl; O—, S—, or N-alkenyl; O—, S— or N-alkynyl; or O-alkyl-O-alkyl, wherein the alkyl, alkenyl, and alkynyl may be substituted or unsubstituted C₁₋₁₀alkyl or C₂₋₁₀alkenyl, and C₂₋₁₀alkynyl. Exemplary 2′ sugar modifications also include, but are not limited to, —O[(CH₂)ₙO]ₘCH₃, —O(CH₂)ₙOCH₃, —O(CH₂)ₙNH₂, —O(CH₂)ₙCH₃, —O(CH₂)ₙ—ONH₂, and —O(CH₂)ₙON[(CH₂)ₙCH₃)]₂, where n and m, independently, are from 1 to about 10. Other modifications at the 2′ position include, but are not limited to, C₁₋₁₀alkyl, substituted lower alkyl, alkaryl, aralkyl, O-alkaryl or O-aralkyl, SH, SCH₃, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving the pharmacokinetic properties of an oligonucleotide, or a group for improving the pharmacodynamic properties of an oligonucleotide, and other substituents having similar properties. Similar modifications may also be made at other positions on the sugar, particularly the 3′ position of the sugar on the 3′ terminal nucleotide or in 2′-5′ linked oligonucleotides and the 5′ position of 5′ terminal nucleotide. Modified sugars can also include those that contain modifications at the bridging ring oxygen, such as CH₂ and S. Nucleotide sugar analogs can also have sugar mimetics, such as cyclobutyl moieties in place of the pentofuranosyl sugar.

Nucleotide analogs can also be modified at the phosphate moiety. Modified phosphate moieties include, but are not limited to, those that can be modified so that the linkage between two nucleotides contains a phosphorothioate, chiral phosphorothioate, phosphorodithioate, phosphotriester, aminoalkylphosphotriester, methyl and other alkyl phosphonates including 3′-alkylene phosphonate and chiral phosphonates, phosphinates, phosphoramidates including 3′-amino phosphoramidate and aminoalkylphosphoramidates, thionophosphoramidates, thionoalkylphosphonates, thionoalkylphosphotriesters, and boranophosphates. These phosphate or modified phosphate linkage between two nucleotides can be through a 3′-5′ linkage or a 2′-5′ linkage, and the linkage can contain inverted polarity such as 3′-5′ to 5′-3′ or 2′-5′ to 5′-2′. Various salts, mixed salts, and free acid forms are also included. Nucleotide substitutes also include peptide nucleic acids (PNAs).

In some embodiments, the antisense nucleic acid molecules are gapmers, whereby the first one to seven nucleotides at the 5′ and 3′ ends each have 2′-methoxyethyl (2′-MOE) modifications. In some embodiments, the first five nucleotides at the 5′ and 3′ ends each have 2′-MOE modifications. In some embodiments, the first one to seven nucleotides at the 5′ and 3′ ends are RNA nucleotides. In some embodiments, the first five nucleotides at the 5′ and 3′ ends are RNA nucleotides. In some embodiments, each of the backbone linkages between the nucleotides is a phosphorothioate linkage.

In some embodiments, the siRNA molecules have termini modifications. In some embodiments, the 5′ end of the antisense strand is phosphorylated. In some embodiments, 5′-phosphate analogs that cannot be hydrolyzed, such as 5′-(E)-vinyl-phosphonate are used.

In some embodiments, the siRNA molecules have backbone modifications. In some embodiments, the modified phosphodiester groups that link consecutive ribose nucleosides have been shown to enhance the stability and in vivo bioavailability of siRNAs The non-ester groups (-OH, =O) of the phosphodiester linkage can be replaced with sulfur, boron, or acetate to give phosphorothioate, boranophosphate, and phosphonoacetate linkages. In addition, substituting the phosphodiester group with a phosphotriester can facilitate cellular uptake of siRNAs and retention on serum components by eliminating their negative charge. In some embodiments, the siRNA molecules have sugar modifications. In some embodiments, the sugars are deprotonated (reaction catalyzed by exo- and endonucleases) whereby the 2′-hydroxyl can act as a nucleophile and attack the adjacent phosphorous in the phosphodiester bond. Such alternatives include 2′-O-methyl, 2′-O-methoxyethyl, and 2′-fluoro modifications.

In some embodiments, the siRNA molecules have base modifications. In some embodiments, the bases can be substituted with modified bases such as pseudouridine, 5′-methylcytidine, N6-methyladenosine, inosine, and N7-methylguanosine.

In some embodiments, the siRNA molecules are conjugated to lipids. Lipids can be conjugated to the 5′ or 3′ termini of siRNA to improve their in vivo bioavailability by allowing them to associate with serum lipoproteins. Representative lipids include, but are not limited to, cholesterol and vitamin E, and fatty acids, such as palmitate and tocopherol.

In some embodiments, a representative siRNA has the following formula:
Sense: mN*mN*/i2FN/mN/i2FN/mN/i2FN/mN/i2FN/mN/i2FN/mN/i2FN/mN/i2FN/mN/i2FN/*mN*/32FN/
Antisense: /52FN/*/i2FN/*mN/i2FN/mN/i2FN/mN/i2FN/mN/i2FN/mN/i2FN/mN/i2FN/mN/i2FN/mN/i2FN/mN*N*N
wherein: “N” is the base; “2F” is a 2′—F modification; “m” is a 2′-O-methyl modification, “I” is an internal base; and “*” is a phosphorothioate backbone linkage.

In any of the embodiments described herein, the inhibitory nucleic acid molecules may be administered, for example, as one to two hour i.v. infusions or s.c. injections. In any of the embodiments described herein, the inhibitory nucleic acid molecules may be administered at dose levels that range from about 50 mg to about 900 mg, from about 100 mg to about 800 mg, from about 150 mg to about 700 mg, or from about 175 to about 640 mg (2.5 to 9.14 mg/kg; 92.5 to 338 mg/m² - based on an assumption of a body weight of 70 kg and a conversion of mg/kg to mg/m² dose levels based on a mg/kg dose multiplier value of 37 for humans).

The present disclosure also provides vectors comprising any one or more of the inhibitory nucleic acid molecules. In some embodiments, the vectors comprise any one or more of the inhibitory nucleic acid molecules and a heterologous nucleic acid. The vectors can be viral or nonviral vectors capable of transporting a nucleic acid molecule. In some embodiments, the vector is a plasmid or cosmid (such as, for example, a circular double-stranded DNA into which additional DNA segments can be ligated). In some embodiments, the vector is a viral vector, wherein additional DNA segments can be ligated into the viral genome. Expression vectors include, but are not limited to, plasmids, cosmids, retroviruses, adenoviruses, adeno-associated viruses (AAV), plant viruses such as cauliflower mosaic virus and tobacco mosaic virus, yeast artificial chromosomes (YACs), Epstein-Barr (EBV)-derived episomes, and other expression vectors known in the art.

The present disclosure also provides compositions comprising any one or more of the inhibitory nucleic acid molecules. In some embodiments, the composition is a pharmaceutical composition. In some embodiments, the compositions comprise a carrier and/or excipient. Examples of carriers include, but are not limited to, poly(lactic acid) (PLA) microspheres, poly(D,L-lactic-coglycolic-acid) (PLGA) microspheres, liposomes, micelles, inverse micelles, lipid cochleates, and lipid microtubules. A carrier may comprise a buffered salt solution such as PBS, HBSS, etc.

In some embodiments, the ITGA1 inhibitor comprises a nuclease agent that induces one or more nicks or double-strand breaks at a recognition sequence(s) or a DNA-binding protein that binds to a recognition sequence within an ITGA1 genomic nucleic acid molecule. The recognition sequence can be located within a coding region of the ITGA1 gene, or within regulatory regions that influence the expression of the gene. A recognition sequence of the DNA-binding protein or nuclease agent can be located in an intron, an exon, a promoter, an enhancer, a regulatory region, or any non-protein coding region. The recognition sequence can include or be proximate to the start codon of the ITGA1 gene. For example, the recognition sequence can be located about 10, about 20, about 30, about 40, about 50, about 100, about 200, about 300, about 400, about 500, or about 1,000 nucleotides from the start codon. As another example, two or more nuclease agents can be used, each targeting a nuclease recognition sequence including or proximate to the start codon. As another example, two nuclease agents can be used, one targeting a nuclease recognition sequence including or proximate to the start codon, and one targeting a nuclease recognition sequence including or proximate to the stop codon, wherein cleavage by the nuclease agents can result in deletion of the coding region between the two nuclease recognition sequences. Any nuclease agent that induces a nick or double-strand break into a desired recognition sequence can be used in the methods and compositions disclosed herein. Any DNA-binding protein that binds to a desired recognition sequence can be used in the methods and compositions disclosed herein.

Suitable nuclease agents and DNA-binding proteins for use herein include, but are not limited to, zinc finger protein or zinc finger nuclease (ZFN) pair, Transcription Activator-Like Effector (TALE) protein or Transcription Activator-Like Effector Nuclease (TALEN), or Clustered Regularly Interspersed Short Palindromic Repeats (CRISPR)/CRISPR-associated (Cas) systems. The length of the recognition sequence can vary, and includes, for example, recognition sequences that are about 30-36 bp for a zinc finger protein or ZFN pair, about 15-18 bp for each ZFN, about 36 bp for a TALE protein or TALEN, and about 20 bp for a CRISPR/Cas guide RNA.

In some embodiments, CRISPR/Cas systems can be used to modify an ITGA1 genomic nucleic acid molecule within a cell. The methods and compositions disclosed herein can employ CRISPR-Cas systems by utilizing CRISPR complexes (comprising a guide RNA (gRNA) complexed with a Cas protein) for site-directed cleavage of ITGA1 nucleic acid molecules.

Cas proteins generally comprise at least one RNA recognition or binding domain that can interact with gRNAs. Cas proteins can also comprise nuclease domains (such as, for example, DNase or RNase domains), DNA binding domains, helicase domains, protein-protein interaction domains, dimerization domains, and other domains. Suitable Cas proteins include, for example, a wild type Cas9 protein and a wild type Cpf1 protein (such as, for example, FnCpf1). A Cas protein can have full cleavage activity to create a double-strand break in an ITGA1 genomic nucleic acid molecule or it can be a nickase that creates a single-strand break in an ITGA1 genomic nucleic acid molecule. Additional examples of Cas proteins include, but are not limited to, Cas1, Cas1B, Cas2, Cas3, Cas4, Cas5, Cas5e (CasD), Cas6, Cas6e, Cas6f, Cas7, Cas8a1, Cas8a2, Cas8b, Cas8c, Cas9 (Csn1 or Csx12), Cas10, Cas10d, CasF, CasG, CasH, Csy1, Csy2, Csy3, Cse1 (CasA), Cse2 (CasB), Cse3 (CasE), Cse4 (CasC), Csc1, Csc2, Csa5, Csn2, Csm2, Csm3, Csm4, Csm5, Csm6, Cmr1 , Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and Cu1966, and homologs or modified versions thereof. In some embodiments, a Cas system, such as Cas12a, can have multiple gRNAs encoded into a single crRNA. Cas proteins can also be operably linked to heterologous polypeptides as fusion proteins. For example, a Cas protein can be fused to a cleavage domain, an epigenetic modification domain, a transcriptional activation domain, or a transcriptional repressor domain. Cas proteins can be provided in any form. For example, a Cas protein can be provided in the form of a protein, such as a Cas protein complexed with a gRNA. Alternately, a Cas protein can be provided in the form of a nucleic acid molecule encoding the Cas protein, such as an RNA or DNA.

In some embodiments, targeted genetic modifications of an ITGA1 genomic nucleic acid molecules can be generated by contacting a cell with a Cas protein and one or more gRNAs that hybridize to one or more gRNA recognition sequences within a target genomic locus in the ITGA1 genomic nucleic acid molecule. For example, a gRNA recognition sequence can be located within a region of SEQ ID NO:1. The gRNA recognition sequence can also include or be proximate to a position corresponding to a variant position. For example, the gRNA recognition sequence can be located from about 1000, from about 500, from about 400, from about 300, from about 200, from about 100, from about 50, from about 45, from about 40, from about 35, from about 30, from about 25, from about 20, from about 15, from about 10, or from about 5 nucleotides of a variant position. The gRNA recognition sequence can include or be proximate to the start codon of an ITGA1 genomic nucleic acid molecule or the stop codon of an ITGA1 genomic nucleic acid molecule. For example, the gRNA recognition sequence can be located from about 10, from about 20, from about 30, from about 40, from about 50, from about 100, from about 200, from about 300, from about 400, from about 500, or from about 1,000 nucleotides of the start codon or the stop codon.

The gRNA recognition sequences within a target genomic locus in an ITGA1 genomic nucleic acid molecule are located near a Protospacer Adjacent Motif (PAM) sequence, which is a 2-6 base pair DNA sequence immediately following the DNA sequence targeted by the Cas9 nuclease. The canonical PAM is the sequence 5′-NGG-3′ where “N” is any nucleobase followed by two guanine (“G”) nucleobases. gRNAs can transport Cas9 to anywhere in the genome for gene editing, but no editing can occur at any site other than one at which Cas9 recognizes PAM. In addition, 5′-NGA-3′ can be a highly efficient non-canonical PAM for human cells. Generally, the PAM is about 2 to about 6 nucleotides downstream of the DNA sequence targeted by the gRNA. The PAM can flank the gRNA recognition sequence. In some embodiments, the gRNA recognition sequence can be flanked on the 3′ end by the PAM. In some embodiments, the gRNA recognition sequence can be flanked on the 5′ end by the PAM. For example, the cleavage site of Cas proteins can be about 1 to about 10 base pairs, about 2 to about 5 base pairs, or 3 base pairs upstream or downstream of the PAM sequence. In some embodiments (such as when Cas9 from S. pyogenes or a closely related Cas9 is used), the PAM sequence of the non-complementary strand can be 5′-NGG-3′, where N is any DNA nucleotide and is immediately 3′ of the gRNA recognition sequence of the non-complementary strand of the target DNA. As such, the PAM sequence of the complementary strand would be 5′—CCN—3′, where N is any DNA nucleotide and is immediately 5′ of the gRNA recognition sequence of the complementary strand of the target DNA.

A gRNA is an RNA molecule that binds to a Cas protein and targets the Cas protein to a specific location within an ITGA1 genomic nucleic acid molecule. An exemplary gRNA is a gRNA effective to direct a Cas enzyme to bind to or cleave an ITGA1 genomic nucleic acid molecule, wherein the gRNA comprises a DNA-targeting segment that hybridizes to a gRNA recognition sequence within the ITGA1 genomic nucleic acid molecule that includes or is proximate to a position corresponding to a variant position. For example, a gRNA can be selected such that it hybridizes to a gRNA recognition sequence that is located about 5, about 10, about 15, about 20, about 25, about 30, about 35, about 40, about 45, about 50, about 100, about 200, about 300, about 400, about 500, or about 1,000 nucleotides from a variant position. Other exemplary gRNAs comprise a DNA-targeting segment that hybridizes to a gRNA recognition sequence present within an ITGA1 genomic nucleic acid molecule that includes or is proximate to the start codon or the stop codon. For example, a gRNA can be selected such that it hybridizes to a gRNA recognition sequence that is located about 5, about 10, about 15, about 20, about 25, about 30, about 35, about 40, about 45, about 50, about 100, about 200, about 300, about 400, about 500, or about 1,000 nucleotides of the start codon or located about 5, about 10, about 15, about 20, about 25, about 30, about 35, about 40, about 45, about 50, about 100, about 200, about 300, about 400, about 500, or about 1,000 nucleotides of the stop codon. Suitable gRNAs can comprise from about 17 to about 25 nucleotides, from about 17 to about 23 nucleotides, from about 18 to about 22 nucleotides, or from about 19 to about 21 nucleotides. In some embodiments, the gRNAs can comprise 20 nucleotides.

Examples of suitable gRNA recognition sequences located within the ITGA1 reference gene are set forth in Table 1 as SEQ ID NOs:12-31.

TABLE 1

Guide RNA Recognition Sequences Near ITGA1 Variation(s)

| Strand | gRNA Recognition Sequence | SEQ ID NO: |
|---|---|---|
| - | TGGCCTGTATGATTGTACCG | 12 |
| - | TGCAACAAGTACCTCTTCGG | 13 |
| + | ACAGCGTATTCCATCAGGTG | 14 |
| + | CACAGCGTATTCCATCAGGT | 15 |
| + | TAAGTATTCTTCCACCGAAG | 16 |
| + | ATTCTTGGCAGCTATAACCG | 17 |
| + | TGTGTAAAGTTGGATCTACC | 18 |
| - | GGTAGATCCAACTTTACACA | 19 |
| + | GAAAACAAAAATGAGCCATG | 20 |
| + | TTCCAACAGTATTTACCCAT | 21 |
| + | AACAACAACTGACATTGACA | 22 |
| + | TAAGCACTCCTTCTACATGT | 23 |
| + | TCGGTACAATCATACAGGCC | 24 |
| - | TTAGTGAATCTAGGGTGACA | 25 |
| + | GAGGCATTCACGGAAGCCCG | 26 |
| - | AATTCACGACTTGAAATGTG | 27 |
| + | AAGAAAAACTGCCATATGGA | 28 |
| - | TAAAATCTGTACCTTGTACA | 29 |
| + | GAGCAGTAGGAGCCTATGAT | 30 |
| + | GTTTGAATATCAAATGAGCC | 31 |

The Cas protein and the gRNA form a complex, and the Cas protein cleaves the target ITGA1 genomic nucleic acid molecule. The Cas protein can cleave the nucleic acid molecule at a site within or outside of the nucleic acid sequence present in the target ITGA1 genomic nucleic acid molecule to which the DNA-targeting segment of a gRNA will bind. For example, formation of a CRISPR complex (comprising a gRNA hybridized to a gRNA recognition sequence and complexed with a Cas protein) can result in cleavage of one or both strands in or near (such as, for example, within 1, 2, 3, 4, 5, 6, 7, 8, 9, 10, 20, 50, or more base pairs from) the nucleic acid sequence present in the ITGA1 genomic nucleic acid molecule to which a DNA-targeting segment of a gRNA will bind.

Such methods can result, for example, in an ITGA1 genomic nucleic acid molecule in which a region of SEQ ID NO:1 is disrupted, the start codon is disrupted, the stop codon is disrupted, or the coding sequence is disrupted or deleted. Optionally, the cell can be further contacted with one or more additional gRNAs that hybridize to additional gRNA recognition sequences within the target genomic locus in the ITGA1 genomic nucleic acid molecule. By contacting the cell with one or more additional gRNAs (such as, for example, a second gRNA that hybridizes to a second gRNA recognition sequence), cleavage by the Cas protein can create two or more double-strand breaks or two or more single-strand breaks.

In some embodiments, the ITGA1 inhibitor comprises a small molecule. In some embodiments, the ITGA1 inhibitor is obtustatin.

In some embodiments, the methods of treatment further comprise detecting the presence or absence of an ITGA1 variant nucleic acid in a biological sample obtained from the subject.

The present disclosure also provides methods of treating a subject with a lung disease therapeutic agent. In some embodiments, the subject has a lung disease. In some embodiments, the subject has an increased risk of developing a lung disease. In some embodiments, the methods comprise determining whether the subject has an ITGA1 variant nucleic acid molecule by obtaining or having obtained a biological sample from the subject, and performing or having performed a sequence analysis on the biological sample to determine if the subject has a genotype comprising the ITGA1 variant nucleic acid molecule. When the subject is ITGA1 reference, the lung disease therapeutic agent is administered or continued to be administered to the subject in a standard dosage amount, and an ITGA1 inhibitor is administered to the subject. When the subject is heterozygous for an ITGA1 variant nucleic acid molecule, the lung disease therapeutic agent is administered or continued to be administered to the subject in an amount that is the same as or less than a standard dosage amount, and an ITGA1 inhibitor is administered to the subject. The presence of a genotype having the ITGA1 variant nucleic acid molecule indicates the subject has a decreased risk of developing a lung disease. In some embodiments, the subject is ITGA1 reference. In some embodiments, the subject is heterozygous for the ITGA1 variant nucleic acid molecule.

For subjects that are genotyped or determined to be either ITGA1 reference or heterozygous for the ITGA1 variant nucleic acid molecule, such subjects can be treated with an ITGA1 inhibitor, as described herein.

Detecting the presence or absence of an ITGA1 variant nucleic acid molecule in a biological sample from a subject and/or determining whether a subject has an ITGA1 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the ITGA1 variant nucleic acid molecule can be present within a cell obtained from the subject.

In some embodiments, when the subject is ITGA1 reference, the subject is also administered a lung disease therapeutic agent in a standard dosage amount. In some embodiments, when the subject is heterozygous for an ITGA1 variant nucleic acid molecule, the subject is also administered a lung disease therapeutic agent in a dosage amount that is the same as or less than a standard dosage amount.

In some embodiments, the treatment methods further comprise detecting the presence or absence of an ITGA1 predicted loss-of-function polypeptide in a biological sample from the subject. In some embodiments, when the subject does not have an ITGA1 predicted loss-of-function polypeptide, the subject is also administered a lung disease therapeutic agent in a standard dosage amount. In some embodiments, when the subject has an ITGA1 predicted loss-of-function polypeptide, the subject is also administered a lung disease therapeutic agent in a dosage amount that is the same as or less than a standard dosage amount.

The present disclosure also provides methods of treating a subject with a lung disease therapeutic agent. In some embodiments, the subject has a lung disease. In some embodiments, the subject has an increased risk of developing a lung disease. In some embodiments, the method comprises determining whether the subject has an ITGA1 predicted loss-of-function polypeptide by obtaining or having obtained a biological sample from the subject, and performing or having performed an assay on the biological sample to determine if the subject has an ITGA1 predicted loss-of-function polypeptide. When the subject does not have an ITGA1 predicted loss-of-function polypeptide, the lung disease therapeutic agent is administered or continued to be administered to the subject in a standard dosage amount, and an ITGA1 inhibitor is administered to the subject. When the subject has an ITGA1 predicted loss-of-function polypeptide, the lung disease therapeutic agent is administered or continued to be administered to the subject in an amount that is the same as or less than a standard dosage amount, and an ITGA1 inhibitor is administered to the subject. The presence of an ITGA1 predicted loss-of-function polypeptide indicates the subject has a decreased risk of developing a lung disease. In some embodiments, the subject has an ITGA1 predicted loss-of-function polypeptide. In some embodiments, the subject does not have an ITGA1 predicted loss-of-function polypeptide.

Detecting the presence or absence of an ITGA1 predicted loss-of-function polypeptide in a biological sample from a subject and/or determining whether a subject has an ITGA1 predicted loss-of-function polypeptide can be carried out by any of the methods described herein. In some embodiments, these methods can be carried out in vitro. In some embodiments, these methods can be carried out in situ. In some embodiments, these methods can be carried out in vivo. In any of these embodiments, the ITGA1 predicted loss-of-function polypeptide can be present within a cell obtained from the subject.

Examples of lung disease therapeutic agents include, but are not limited to: nintedanib, pirfenidone, prednisone, azathioprine, cyclophosphamide, mycophenolate, mofetil, rituximab, tacrolimus, cotrimoxazole, lebrikizumab, nandrolone decanoate, sirolimus, thalidomide, and pomalidomide.

In some embodiments, the dose of the lung disease therapeutic agents can be reduced by about 10%, by about 20%, by about 30%, by about 40%, by about 50%, by about 60%, by about 70%, by about 80%, or by about 90% for subjects that are heterozygous for an ITGA1 variant nucleic acid molecule (i.e., a less than the standard dosage amount) compared to subjects that are ITGA1 reference (who may receive a standard dosage amount). In some embodiments, the dose of the lung disease therapeutic agent can be reduced by about 10%, by about 20%, by about 30%, by about 40%, or by about 50%. In addition, the dose of lung disease therapeutic agents in subjects that are heterozygous for an ITGA1 variant nucleic acid molecule can be administered less frequently compared to subjects that are ITGA1 reference.

Administration of the lung disease therapeutic agents and/or ITGA1 inhibitors can be repeated, for example, after one day, two days, three days, five days, one week, two weeks, three weeks, one month, five weeks, six weeks, seven weeks, eight weeks, two months, or three months. The repeated administration can be at the same dose or at a different dose. The administration can be repeated once, twice, three times, four times, five times, six times, seven times, eight times, nine times, ten times, or more. For example, according to certain dosage regimens a subject can receive therapy for a prolonged period of time such as, for example, 6 months, 1 year, or more. In addition, the lung disease therapeutic agents and/or ITGA1 inhibitors can be administered sequentially or at the same time. In addition, the lung disease therapeutic agents and/or ITGA1 inhibitors can be administered in separate compositions or can be administered together in the same composition.

Administration of the lung disease therapeutic agents and/or ITGA1 inhibitors can occur by any suitable route including, but not limited to, parenteral, intravenous, oral, subcutaneous, intra-arterial, intracranial, intrathecal, intraperitoneal, topical, intranasal, or intramuscular. Pharmaceutical compositions for administration are desirably sterile and substantially isotonic and manufactured under GMP conditions. Pharmaceutical compositions can be provided in unit dosage form (i.e., the dosage for a single administration). Pharmaceutical compositions can be formulated using one or more physiologically and pharmaceutically acceptable carriers, diluents, excipients or auxiliaries. The formulation depends on the route of administration chosen. The term “pharmaceutically acceptable” means that the carrier, diluent, excipient, or auxiliary is compatible with the other ingredients of the formulation and not substantially deleterious to the recipient thereof.

The terms “treat”, “treating”, and “treatment” and “prevent”, “preventing”, and “prevention” as used herein, refer to eliciting the desired biological response, such as a therapeutic and prophylactic effect, respectively. In some embodiments, a therapeutic effect comprises one or more of a decrease/reduction in a lung disease, a decrease/reduction in the severity of a lung disease (such as, for example, a reduction or inhibition of development of a lung disease), a decrease/reduction in symptoms and lung disease-related effects, delaying the onset of symptoms and lung disease-related effects, reducing the severity of symptoms of lung disease-related effects, reducing the severity of an acute episode, reducing the number of symptoms and lung disease-related effects, reducing the latency of symptoms and lung disease-related effects, an amelioration of symptoms and lung disease-related effects, reducing secondary symptoms, reducing secondary infections, preventing relapse to a lung disease, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics, and/or an increased survival time of the affected host animal, following administration of the agent or composition comprising the agent. A prophylactic effect may comprise a complete or partial avoidance/inhibition or a delay of a lung disease development/progression (such as, for example, a complete or partial avoidance/inhibition or a delay), and an increased survival time of the affected host animal, following administration of a therapeutic protocol. Treatment of a lung disease encompasses the treatment of subjects already diagnosed as having any form of a lung disease at any clinical stage or manifestation, the delay of the onset or evolution or aggravation or deterioration of the symptoms or signs of a lung disease, and/or preventing and/or reducing the severity of a lung disease.

The present disclosure also provides methods of identifying a subject having an increased risk of developing a fibrotic lung disease, pulmonary fibrosis, interstitial lung disease, COPD, or asthma. In some embodiments, the methods comprise determining or having determined the presence or absence of an ITGA1 variant nucleic acid molecule (such as a genomic nucleic acid molecule, mRNA molecule, and/or cDNA molecule) in a biological sample obtained from the subject. When the subject lacks an ITGA1 variant nucleic acid molecule (i.e., the subject is genotypically categorized as ITGA1 reference), then the subject has an increased risk of developing a fibrotic lung disease, pulmonary fibrosis, interstitial lung disease, COPD, or asthma. When the subject has an ITGA1 variant nucleic acid molecule (i.e., the subject is heterozygous or homozygous for an ITGA1 variant nucleic acid molecule), then the subject has a decreased risk of developing a fibrotic lung disease, pulmonary fibrosis, interstitial lung disease, COPD, or asthma compared to a subject that is ITGA1 reference.

Having a single copy of an ITGA1 variant nucleic acid molecule is more protective of a subject from developing a fibrotic lung disease, pulmonary fibrosis, interstitial lung disease, COPD, or asthma than having no copies of an ITGA1 variant nucleic acid molecule. Without intending to be limited to any particular theory or mechanism of action, it is believed that a single copy of an ITGA1 variant nucleic acid molecule (i.e., heterozygous for an ITGA1 variant nucleic acid molecule) is protective of a subject from developing a fibrotic lung disease, pulmonary fibrosis, interstitial lung disease, COPD, or asthma, and it is also believed that having two copies of an ITGA1 variant nucleic acid molecule (i.e., homozygous for an ITGA1 variant nucleic acid molecule) may be more protective of a subject from developing a fibrotic lung disease, pulmonary fibrosis, interstitial lung disease, COPD, or asthma, relative to a subject with a single copy. Thus, in some embodiments, a single copy of an ITGA1 variant nucleic acid molecule may not be completely protective, but instead, may be partially or incompletely protective of a subject from developing a fibrotic lung disease, pulmonary fibrosis, interstitial lung disease, COPD, or asthma. While not desiring to be bound by any particular theory, there may be additional factors or molecules involved in the development of a fibrotic lung disease, pulmonary fibrosis, interstitial lung disease, COPD, or asthma that are still present in a subject having a single copy of an ITGA1 variant nucleic acid molecule, thus resulting in less than complete protection from the development of a fibrotic lung disease, pulmonary fibrosis, interstitial lung disease, COPD, or asthma.

Detecting the presence or absence of an ITGA1 variant nucleic acid molecule in a biological sample from the subject and/or determining whether a subject has an ITGA1 variant nucleic acid molecule can be carried out by any of the methods described herein. In some embodiments, these methods can be carried out in vitro. In some embodiments, these methods can be carried out in situ. In some embodiments, these methods can be carried out in vivo. In any of these embodiments, the ITGA1 variant nucleic acid molecule can be present within a cell obtained from the subject.

In some embodiments, when a subject is identified as having an increased risk of developing a fibrotic lung disease, pulmonary fibrosis, interstitial lung disease, COPD, or asthma, the subject is further treated with a lung disease therapeutic agent and/or an ITGA1 inhibitor, as described herein. For example, when the subject is ITGA1 reference, and therefore has an increased risk of developing a fibrotic lung disease, pulmonary fibrosis, interstitial lung disease, COPD, or asthma, the subject is administered an ITGA1 inhibitor. In some embodiments, such a subject is also administered a lung disease therapeutic agent. In some embodiments, when the subject is heterozygous for an ITGA1 variant nucleic acid molecule, the subject is administered the lung disease therapeutic agent in a dosage amount that is the same as or less than a standard dosage amount, and is also administered an ITGA1 inhibitor. In some embodiments, the subject is ITGA1 reference. In some embodiments, the subject is heterozygous for an ITGA1 variant nucleic acid molecule.

The present disclosure also provides methods of detecting the presence or absence of an ITGA1 variant genomic nucleic acid molecule in a biological sample obtained from a subject, and/or an ITGA1 variant mRNA molecule in a biological sample obtained from a subject, and/or an ITGA1 variant cDNA molecule produced from an mRNA molecule in a biological sample obtained from a subject. It is understood that gene sequences within a population and mRNA molecules encoded by such genes can vary due to polymorphisms such as single-nucleotide polymorphisms. The sequences provided herein for the ITGA1 variant genomic nucleic acid molecule, ITGA1 variant mRNA molecule, and ITGA1 variant cDNA molecule are only exemplary sequences. Other sequences for the ITGA1 variant genomic nucleic acid molecule, variant mRNA molecule, and variant cDNA molecule are also possible.

The biological sample can be derived from any cell, tissue, or biological fluid from the subject. The biological sample may comprise any clinically relevant tissue such as, for example, a bone marrow sample, a tumor biopsy, a fine needle aspirate, or a sample of bodily fluid, such as blood, gingival crevicular fluid, plasma, serum, lymph, ascitic fluid, cystic fluid, or urine. In some embodiments, the biological sample comprises a buccal swab. The biological sample used in the methods disclosed herein can vary based on the assay format, nature of the detection method, and the tissues, cells, or extracts that are used as the sample. A biological sample can be processed differently depending on the assay being employed. For example, when detecting any ITGA1 variant nucleic acid molecule, preliminary processing designed to isolate or enrich the biological sample for the ITGA1 variant nucleic acid molecule can be employed. A variety of techniques may be used for this purpose. When detecting the level of any ITGA1 variant mRNA molecule, different techniques can be used enrich the biological sample with mRNA molecules. Various methods to detect the presence or level of an mRNA molecule or the presence of a particular variant genomic DNA locus can be used.

The present disclosure also provides methods of detecting an ITGA1 variant nucleic acid molecule, or the complement thereof, in a subject. The methods comprise assaying a biological sample obtained from the subject to determine whether a nucleic acid molecule in the biological sample is an ITGA1 variant nucleic acid molecule.

In some embodiments, the ITGA1 variant nucleic acid molecule, or the complement thereof, is a genomic nucleic acid molecule having a nucleotide sequence comprising a nucleotide at a position corresponding to the nucleotide at the variant position of an ITGA1 variant nucleic acid molecule, or the complement thereof. In some embodiments, the ITGA1 variant nucleic acid molecule, is an mRNA molecule having a nucleotide sequence comprising a nucleotide at a position corresponding to the nucleotide at the variant position of an ITGA1 variant nucleic acid molecule, or the complement thereof. In some embodiments, the ITGA1 variant nucleic acid molecule, is a cDNA molecule produced from an mRNA molecule in the biological sample having a nucleotide sequence comprising a nucleotide at a position corresponding to the nucleotide at the variant position of an ITGA1 variant nucleic acid molecule, or the complement thereof.

In some embodiments, the biological sample comprises a cell or cell lysate. Such methods can further comprise, for example, obtaining a biological sample from the subject comprising an ITGA1 genomic nucleic acid molecule or mRNA molecule, and if mRNA, optionally reverse transcribing the mRNA into cDNA. Such assays can comprise, for example determining the identity of these positions of the particular ITGA1 nucleic acid molecule. In some embodiments, the method is an in vitro method.

In some embodiments, the determining step, detecting step, or sequence analysis comprises sequencing at least a portion of the nucleotide sequence of the ITGA1 genomic nucleic acid molecule, the ITGA1 mRNA molecule, or the ITGA1 cDNA molecule produced from the mRNA molecule in the biological sample, wherein the sequenced portion comprises one or more variations that cause a loss-of-function (partial or complete) or are predicted to cause a loss-of-function (partial or complete).

In some embodiments, the determining step, detecting step, or sequence analysis comprises sequencing at least a portion of: i) the nucleotide sequence of the ITGA1 genomic nucleic acid molecule in the biological sample, wherein the sequenced portion comprises a position corresponding to the variant position of an ITGA1 variant nucleic acid molecule, or the complement thereof; ii) the nucleotide sequence of the ITGA1 mRNA molecule in the biological sample, wherein the sequenced portion comprises a position corresponding to the variant position of an ITGA1 variant nucleic acid molecule, or the complement thereof; and/or iii) the nucleotide sequence of the ITGA1 cDNA molecule produced from the mRNA in the biological sample, wherein the sequenced portion comprises a position corresponding to the variant position of an ITGA1 variant nucleic acid molecule, or the complement thereof. When the sequenced portion of the ITGA1 nucleic acid molecule in the biological sample comprises a nucleotide sequence that comprises a nucleotide at a position corresponding to the nucleotide at the variant position of an ITGA1 variant nucleic acid molecule, then the ITGA1 nucleic acid molecule in the biological sample is an ITGA1 variant nucleic acid molecule.

In some embodiments, the determining step, detecting step, or sequence analysis comprises sequencing at least a portion of the nucleotide sequence of the ITGA1 genomic nucleic acid molecule in the biological sample, wherein the sequenced portion comprises a position corresponding to the variant position of an ITGA1 variant nucleic acid molecule, or the complement thereof. When the sequenced portion of the ITGA1 nucleic acid molecule in the biological sample comprises a nucleotide sequence that comprises a nucleotide at a position corresponding to the nucleotide at the variant position of an ITGA1 variant nucleic acid molecule, then the ITGA1 nucleic acid molecule in the biological sample is an ITGA1 variant genomic nucleic acid molecule.

In some embodiments, the determining step, detecting step, or sequence analysis comprises sequencing at least a portion of the nucleotide sequence of the ITGA1 mRNA molecule in the biological sample, wherein the sequenced portion comprises a position corresponding to the variant position of an ITGA1 variant nucleic acid molecule, or the complement thereof. When the sequenced portion of the ITGA1 mRNA molecule in the biological sample comprises a nucleotide sequence that comprises a nucleotide at a position corresponding to the nucleotide at the variant position of an ITGA1 variant nucleic acid molecule, then the ITGA1 nucleic acid molecule in the biological sample is an ITGA1 variant mRNA molecule.

In some embodiments, the determining step, detecting step, or sequence analysis comprises sequencing at least a portion of the nucleotide sequence of the ITGA1 cDNA molecule produced from the mRNA molecule in the biological sample, wherein the sequenced portion comprises a position corresponding to the variant position of an ITGA1 variant nucleic acid molecule, or the complement thereof. When the sequenced portion of the ITGA1 cDNA molecule in the biological sample comprises a nucleotide sequence that comprises a nucleotide at a position corresponding to the nucleotide at the variant position of an ITGA1 variant nucleic acid molecule, then the ITGA1 nucleic acid molecule in the biological sample is an ITGA1 variant cDNA molecule.

In some embodiments, the determining step, detecting step, or sequence analysis comprises: a) contacting the biological sample with a primer hybridizing to a portion of the nucleotide sequence of the ITGA1: i) genomic nucleic acid molecule, or the complement thereof, that is proximate to a position corresponding to a variant position of an ITGA1 variant nucleic acid molecule, or the complement thereof; ii) mRNA molecule, or the complement thereof, that is proximate to a variant position of an ITGA1 variant nucleic acid molecule, or the complement thereof; and/or iii) cDNA molecule, or the complement thereof, that is proximate to a position corresponding to a variant position of an ITGA1 variant nucleic acid molecule, or the complement thereof; b) extending the primer at least through the position of the nucleotide sequence of the ITGA1: i) genomic nucleic acid molecule, or the complement thereof, corresponding to a variant position of an ITGA1 variant nucleic acid molecule, or the complement thereof; ii) mRNA molecule, or the complement thereof, corresponding to a variant position of an ITGA1 variant nucleic acid molecule, or the complement thereof; and/or iii) cDNA molecule, or the complement thereof, corresponding to a variant position of an ITGA1 variant nucleic acid molecule, or the complement thereof; and c) determining whether the extension product of the primer comprise a nucleotide at a position corresponding to the nucleotide at the variant position of an ITGA1 variant nucleic acid molecule, or the complement thereof.

In some embodiments, the determining step, detecting step, or sequence analysis comprises: a) contacting the biological sample with a primer hybridizing to a portion of the nucleotide sequence of the ITGA1 genomic nucleic acid molecule, or the complement thereof, that is proximate to a position corresponding to a variant position of an ITGA1 variant nucleic acid molecule, or the complement thereof; b) extending the primer at least through the position of the nucleotide sequence of the ITGA1 genomic nucleic acid molecule, or the complement thereof, corresponding to a variant position of an ITGA1 variant nucleic acid molecule, or the complement thereof; and c) determining whether the extension product of the primer comprises a nucleotide at a position corresponding to the nucleotide at the variant position of an ITGA1 variant nucleic acid molecule, or the complement thereof.

In some embodiments, the determining step, detecting step, or sequence analysis comprises: a) contacting the biological sample with a primer hybridizing to a portion of the nucleotide sequence of the ITGA1 mRNA molecule, or the complement thereof, that is proximate to a position corresponding to a variant position of an ITGA1 variant nucleic acid molecule, or the complement thereof; b) extending the primer at least through the position of the nucleotide sequence of the ITGA1 mRNA molecule corresponding to a variant position of an ITGA1 variant nucleic acid molecule, or the complement thereof; and c) determining whether the extension product of the primer comprises a nucleotide at a position corresponding to the nucleotide at the variant position of an ITGA1 variant nucleic acid molecule, or the complement thereof.

In some embodiments, the determining step, detecting step, or sequence analysis comprises: a) contacting the biological sample with a primer hybridizing to a portion of the nucleotide sequence of the ITGA1 cDNA molecule, or the complement thereof, that is proximate to a position corresponding to a variant position of an ITGA1 variant nucleic acid molecule, or the complement thereof; b) extending the primer at least through the position of the nucleotide sequence of the ITGA1 cDNA molecule corresponding to a variant position of an ITGA1 variant nucleic acid molecule, or the complement thereof; and c) determining whether the extension product of the primer comprises a nucleotide at a position corresponding to the nucleotide at the variant position of an ITGA1 variant nucleic acid molecule, or the complement thereof.

In some embodiments, the entire nucleic acid molecule is sequenced. In some embodiments, only an ITGA1 genomic nucleic acid molecule is analyzed. In some embodiments, only an ITGA1 mRNA is analyzed. In some embodiments, only an ITGA1 cDNA obtained from ITGA1 mRNA is analyzed.

In some embodiments, the determining step, detecting step, or sequence analysis comprises: a) amplifying at least a portion of the ITGA1 nucleic acid molecule, or the complement thereof, in the biological sample, wherein the amplified portion comprise a nucleotide at a position corresponding to the nucleotide at the variant position of an ITGA1 variant nucleic acid molecule, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a nucleotide at a position corresponding to the nucleotide at the variant position of an ITGA1 variant nucleic acid molecule, or the complement thereof.

In some embodiments, the determining step, detecting step, or sequence analysis comprises: a) amplifying at least a portion of the ITGA1 genomic nucleic acid molecule, or the complement thereof, in the biological sample, wherein the portion comprises a nucleotide at a position corresponding to the nucleotide at the variant position of an ITGA1 variant nucleic acid molecule, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a nucleotide at a position corresponding to the nucleotide at the variant position of an ITGA1 variant nucleic acid molecule, or the complement thereof; and d) detecting the detectable label.

In some embodiments, the determining step, detecting step, or sequence analysis comprises: a) amplifying at least a portion of the ITGA1 mRNA molecule, or the complement thereof, in the biological sample, wherein the portion comprises a nucleotide at a position corresponding to the nucleotide at the variant position of an ITGA1 variant nucleic acid molecule, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a nucleotide at a position corresponding to the nucleotide at the variant position of an ITGA1 variant nucleic acid molecule, or the complement thereof; and d) detecting the detectable label.

In some embodiments, the determining step, detecting step, or sequence analysis comprises: a) amplifying at least a portion of the ITGA1 cDNA molecule, or the complement thereof, in the biological sample, wherein the portion comprises a nucleotide at a position corresponding to the nucleotide at the variant position of an ITGA1 variant nucleic acid molecule, or the complement thereof; b) labeling the amplified nucleic acid molecule with a detectable label; c) contacting the labeled nucleic acid molecule with a support comprising an alteration-specific probe, wherein the alteration-specific probe comprises a nucleotide sequence which hybridizes under stringent conditions to the nucleic acid sequence of the amplified nucleic acid molecule comprising a nucleotide at a position corresponding to the nucleotide at the variant position of an ITGA1 variant nucleic acid molecule, or the complement thereof; and d) detecting the detectable label.

In some embodiments, the nucleic acid molecule is mRNA and the determining step further comprises reverse-transcribing the mRNA into a cDNA prior to the amplifying step.

In some embodiments, the determining step, detecting step, or sequence analysis comprises: contacting the ITGA1 nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the ITGA1 nucleic acid molecule, or the complement thereof, comprising a nucleotide at a position corresponding to the nucleotide at the variant position of an ITGA1 variant nucleic acid molecule, or the complement thereof; and detecting the detectable label.

In some embodiments, the determining step, detecting step, or sequence analysis comprises: contacting the ITGA1 genomic nucleic acid molecule, or the complement thereof, in the biological sample with an alteration-specific probe comprising a detectable label, wherein the alteration-specific probe comprises a nucleotide sequence which hybridizes under stringent conditions to the nucleotide sequence of the ITGA1 genomic nucleic acid molecule, or the complement thereof, comprising a nucleotide at a position corresponding to the nucleotide at the variant position of an ITGA1 variant nucleic acid molecule, or the complement thereof; and detecting the detectable label.

In some embodiments, the determining step, detecting step, or sequence analysis comprises: contacting the ITGA1 mRNA molecule, or the complement thereof, in the biological sample with an alteration-specific probe comprising a detectable label, wherein the alteration-specific probe comprises a nucleotide sequence which hybridizes under stringent conditions to the nucleotide sequence of the ITGA1 mRNA molecule, or the complement thereof, comprising a nucleotide at a position corresponding to the nucleotide at the variant position of an ITGA1 variant nucleic acid molecule, or the complement thereof; and detecting the detectable label.

In some embodiments, the determining step, detecting step, or sequence analysis comprises: contacting the ITGA1 cDNA molecule, or the complement thereof, produced from an mRNA molecule in the biological sample with an alteration-specific probe comprising a detectable label, wherein the alteration-specific probe comprises a nucleotide sequence which hybridizes under stringent conditions to the nucleotide sequence of the ITGA1 cDNA molecule, or the complement thereof, comprising a nucleotide at a position corresponding to the nucleotide at the variant position of an ITGA1 variant nucleic acid molecule, or the complement thereof; and detecting the detectable label.

In some embodiments, the ITGA1 nucleic acid molecule is present within a cell obtained from the subject.

Alteration-specific polymerase chain reaction techniques can be used to detect mutations such as SNPs in a nucleic acid sequence. Alteration-specific primers can be used because the DNA polymerase will not extend when a mismatch with the template is present.

In some embodiments, the determining step, detecting step, or sequence analysis comprises contacting the biological sample with a primer or probe, such as an alteration-specific primer or alteration-specific probe, that specifically hybridizes to an ITGA1 variant genomic sequence, variant mRNA sequence, or variant cDNA sequence and not the corresponding ITGA1 reference sequence under stringent conditions, and determining whether hybridization has occurred.

In some embodiments, the assay comprises RNA sequencing (RNA-Seq). In some embodiments, the assays also comprise reverse transcribing mRNA into cDNA, such as by the reverse transcriptase polymerase chain reaction (RT-PCR).

In some embodiments, the methods utilize probes and primers of sufficient nucleotide length to bind to the target nucleotide sequence and specifically detect and/or identify a polynucleotide comprising an ITGA1 variant genomic nucleic acid molecule, variant mRNA molecule, or variant cDNA molecule. The hybridization conditions or reaction conditions can be determined by the operator to achieve this result. The nucleotide length may be any length that is sufficient for use in a detection method of choice, including any assay described or exemplified herein. Such probes and primers can hybridize specifically to a target nucleotide sequence under high stringency hybridization conditions. Probes and primers may have complete nucleotide sequence identity of contiguous nucleotides within the target nucleotide sequence, although probes differing from the target nucleotide sequence and that retain the ability to specifically detect and/or identify a target nucleotide sequence may be designed by conventional methods. Probes and primers can have about 80%, about 85%, about 90%, about 91%, about 92%, about 93%, about 94%, about 95%, about 96%, about 97%, about 98%, about 99%, or 100% sequence identity or complementarity with the nucleotide sequence of the target nucleic acid molecule.

In some embodiments, to determine whether an ITGA1 nucleic acid molecule (genomic nucleic acid molecule, mRNA molecule, or cDNA molecule), or complement thereof, within a biological sample comprises a nucleotide sequence comprising a nucleotide at a position corresponding to the nucleotide at the variant position of an ITGA1 variant nucleic acid molecule, the biological sample can be subjected to an amplification method using a primer pair that includes a first primer derived from the 5′ flanking sequence adjacent to a nucleotide at a position corresponding to the nucleotide at the variant position of an ITGA1 variant nucleic acid molecule, and a second primer derived from the 3′ flanking sequence adjacent to a nucleotide at a position corresponding to the nucleotide at the variant position of an ITGA1 variant nucleic acid molecule to produce an amplicon that is indicative of the presence of the SNP at a position corresponding to the variant position of an ITGA1 variant nucleic acid molecule. In some embodiments, the amplicon may range in length from the combined length of the primer pairs plus one nucleotide base pair to any length of amplicon producible by a DNA amplification protocol. This distance can range from one nucleotide base pair up to the limits of the amplification reaction, or about twenty thousand nucleotide base pairs. Optionally, the primer pair flanks a region including a position corresponding to the variant position of an ITGA1 variant nucleic acid molecule and at least 1, 2, 3, 4, 5, 6, 7, 8, 9, 10, or more nucleotides on each side of a position corresponding to the variant position of an ITGA1 variant nucleic acid molecule.

Similar amplicons can be generated from the mRNA and/or cDNA sequences. PCR primer pairs can be derived from a known sequence, for example, by using computer programs intended for that purpose, such as the PCR primer analysis tool in Vector NTI version 10 (Informax Inc., Bethesda Md.); PrimerSelect (DNASTAR Inc., Madison, Wis.); and Primer3 (Version 0.4.0.COPYRGT., 1991, Whitehead Institute for Biomedical Research, Cambridge, Mass.). Additionally, the sequence can be visually scanned and primers manually identified using known guidelines.

Illustrative examples of nucleic acid sequencing techniques include, but are not limited to, chain terminator (Sanger) sequencing and dye terminator sequencing. Other methods involve nucleic acid hybridization methods other than sequencing, including using labeled primers or probes directed against purified DNA, amplified DNA, and fixed cell preparations (fluorescence in situ hybridization (FISH)). In some methods, a target nucleic acid molecule may be amplified prior to or simultaneous with detection. Illustrative examples of nucleic acid amplification techniques include, but are not limited to, polymerase chain reaction (PCR), ligase chain reaction (LCR), strand displacement amplification (SDA), and nucleic acid sequence based amplification (NASBA). Other methods include, but are not limited to, ligase chain reaction, strand displacement amplification, and thermophilic SDA (tSDA).

In hybridization techniques, stringent conditions can be employed such that a probe or primer will specifically hybridize to its target. In some embodiments, a polynucleotide primer or probe under stringent conditions will hybridize to its target sequence to a detectably greater degree than to other non-target sequences, such as, at least 2-fold, at least 3-fold, at least 4-fold, or more over background, including over 10-fold over background. In some embodiments, a polynucleotide primer or probe under stringent conditions will hybridize to its target nucleotide sequence to a detectably greater degree than to other nucleotide sequences by at least 2-fold. In some embodiments, a polynucleotide primer or probe under stringent conditions will hybridize to its target nucleotide sequence to a detectably greater degree than to other nucleotide sequences by at least 3-fold. In some embodiments, a polynucleotide primer or probe under stringent conditions will hybridize to its target nucleotide sequence to a detectably greater degree than to other nucleotide sequences by at least 4-fold. In some embodiments, a polynucleotide primer or probe under stringent conditions will hybridize to its target nucleotide sequence to a detectably greater degree than to other nucleotide sequences by over 10-fold over background. Stringent conditions are sequence-dependent and will be different in different circumstances.

Appropriate stringency conditions which promote DNA hybridization, for example, 6X sodium chloride/sodium citrate (SSC) at about 45° C., followed by a wash of 2× SSC at 50° C., are known or can be found in Current Protocols in Molecular Biology, John Wiley & Sons, N.Y. (1989), 6.3.1-6.3.6. Typically, stringent conditions for hybridization and detection will be those in which the salt concentration is less than about 1.5 M Na⁺ ion, typically about 0.01 to 1.0 M Na⁺ ion concentration (or other salts) at pH 7.0 to 8.3 and the temperature is at least about 30° C. for short probes (such as, for example, 10 to 50 nucleotides) and at least about 60° C. for longer probes (such as, for example, greater than 50 nucleotides). Stringent conditions may also be achieved with the addition of destabilizing agents such as formamide. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The present disclosure also provides methods of detecting the presence of an ITGA1 predicted loss-of-function polypeptide comprising performing an assay on a biological sample obtained from the subject to determine whether an ITGA1 polypeptide in the biological sample contains one or more variations that causes the polypeptide to have a loss-of-function (partial or complete) or predicted loss-of-function (partial or complete). The ITGA1 predicted loss-of-function polypeptide can be any of the ITGA1 predicted loss-of-function polypeptides described herein.

In some embodiments, the methods comprise performing an assay on a biological sample obtained from a subject to determine whether an ITGA1 polypeptide in the biological sample comprises an amino acid sequence comprising a variation encoded by any of the ITGA1 variant nucleic acid molecules described herein.

In some embodiments, the detecting step comprises sequencing at least a portion of the ITGA1 polypeptide that comprises an amino acid sequence comprising a variation encoded by any of the ITGA1 variant nucleic acid molecules described herein

In some embodiments, the detecting step comprises an immunoassay for detecting the presence of an ITGA1 polypeptide that comprises an amino acid sequence comprising a variation encoded by any of the ITGA1 variant nucleic acid molecules described herein.

In some embodiments, when the subject does not have an ITGA1 predicted loss-of-function polypeptide, the subject has an increased risk of developing a fibrotic lung disease, pulmonary fibrosis, interstitial lung disease, COPD, or asthma. In some embodiments, when the subject has an ITGA1 predicted loss-of-function polypeptide, the subject has a decreased risk of developing a fibrotic lung disease, pulmonary fibrosis, interstitial lung disease, COPD, or asthma.

The present disclosure also provides isolated nucleic acid molecules that hybridize to ITGA1 variant genomic nucleic acid molecules, ITGA1 variant mRNA molecules, and/or ITGA1 variant cDNA molecules (such as any of the genomic variant nucleic acid molecules, mRNA variant molecules, and cDNA variant molecules disclosed herein). In some embodiments, such isolated nucleic acid molecules hybridize to ITGA1 variant nucleic acid molecules under stringent conditions. Such nucleic acid molecules can be used, for example, as probes, primers, alteration-specific probes, or alteration-specific primers as described or exemplified herein.

In some embodiments, the isolated nucleic acid molecules hybridize to a portion of the ITGA1 nucleic acid molecule that comprises a nucleotide at a position corresponding to the nucleotide at the variant position of an ITGA1 variant nucleic acid molecule.

In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, at least about 25, at least about 30, at least about 35, at least about 40, at least about 45, at least about 50, at least about 55, at least about 60, at least about 65, at least about 70, at least about 75, at least about 80, at least about 85, at least about 90, at least about 95, at least about 100, at least about 200, at least about 300, at least about 400, at least about 500, at least about 600, at least about 700, at least about 800, at least about 900, at least about 1000, at least about 2000, at least about 3000, at least about 4000, or at least about 5000 nucleotides. In some embodiments, such isolated nucleic acid molecules comprise or consist of at least about 5, at least about 8, at least about 10, at least about 11, at least about 12, at least about 13, at least about 14, at least about 15, at least about 16, at least about 17, at least about 18, at least about 19, at least about 20, at least about 21, at least about 22, at least about 23, at least about 24, or at least about 25 nucleotides. In some embodiments, the isolated nucleic acid molecules comprise or consist of at least about 18 nucleotides. In some embodiments, the isolated nucleic acid molecules comprise or consists of at least about 15 nucleotides. In some embodiments, the isolated nucleic acid molecules consist of or comprise from about 10 to about 35, from about 10 to about 30, from about 10 to about 25, from about 12 to about 30, from about 12 to about 28, from about 12 to about 24, from about 15 to about 30, from about 15 to about 25, from about 18 to about 30, from about 18 to about 25, from about 18 to about 24, or from about 18 to about 22 nucleotides. In some embodiments, the isolated nucleic acid molecules consist of or comprise from about 18 to about 30 nucleotides. In some embodiments, the isolated nucleic acid molecules comprise or consist of at least about 15 nucleotides to at least about 35 nucleotides.

In some embodiments, the isolated nucleic acid molecules hybridize to at least about 15 contiguous nucleotides of a nucleic acid molecule that is at least about 70%, at least about 75%, at least about 80%, at least about 85%, at least about 90%, at least about 95%, at least about 96%, at least about 97%, at least about 98%, at least about 99%, or 100% identical to ITGA1 variant genomic nucleic acid molecules, ITGA1 variant mRNA molecules, and/or ITGA1 variant cDNA molecules. In some embodiments, the isolated nucleic acid molecules consist of or comprise from about 15 to about 100 nucleotides, or from about 15 to about 35 nucleotides. In some embodiments, the isolated nucleic acid molecules consist of or comprise from about 15 to about 100 nucleotides. In some embodiments, the isolated nucleic acid molecules consist of or comprise from about 15 to about 35 nucleotides.

In some embodiments, the isolated alteration-specific probes or alteration-specific primers comprise at least about 15 nucleotides, wherein the alteration-specific probe or alteration-specific primer comprises a nucleotide sequence which is complementary to the nucleotide sequence of a portion of an ITGA1 variant nucleic acid molecule, or the complement thereof. In some embodiments, the portion comprises a position corresponding to the variant position of an ITGA1 variant nucleic acid molecule, or the complement thereof.

In some embodiments, the alteration-specific probes and alteration-specific primers comprise DNA. In some embodiments, the alteration-specific probes and alteration-specific primers comprise RNA.

In some embodiments, the probes and primers described herein (including alteration-specific probes and alteration-specific primers) have a nucleotide sequence that specifically hybridizes to any of the nucleic acid molecules disclosed herein, or the complement thereof. In some embodiments, the probes and primers specifically hybridize to any of the nucleic acid molecules disclosed herein under stringent conditions.

In some embodiments, the primers, including alteration-specific primers, can be used in second generation sequencing or high throughput sequencing. In some instances, the primers, including alteration-specific primers, can be modified. In particular, the primers can comprise various modifications that are used at different steps of, for example, Massive Parallel Signature Sequencing (MPSS), Polony sequencing, and 454 Pyrosequencing. Modified primers can be used at several steps of the process, including biotinylated primers in the cloning step and fluorescently labeled primers used at the bead loading step and detection step. Polony sequencing is generally performed using a paired-end tags library wherein each molecule of DNA template is about 135 bp in length. Biotinylated primers are used at the bead loading step and emulsion PCR. Fluorescently labeled degenerate nonamer oligonucleotides are used at the detection step. An adaptor can contain a 5′-biotin tag for immobilization of the DNA library onto streptavidin-coated beads.

The probes and primers described herein can be used to detect a nucleotide variation within any of the ITGA1 variant genomic nucleic acid molecules, ITGA1 variant mRNA molecules, and/or ITGA1 variant cDNA molecules disclosed herein. The primers described herein can be used to amplify the ITGA1 variant genomic nucleic acid molecules, ITGA1 variant mRNA molecules, or ITGA1 variant cDNA molecules, or a fragment thereof.

The present disclosure also provides pairs of primers comprising any of the primers described above.

In the context of the present disclosure “specifically hybridizes” means that the probe or primer (such as, for example, the alteration-specific probe or alteration-specific primer) does not hybridize to a nucleic acid sequence encoding an ITGA1 reference genomic nucleic acid molecule, an ITGA1 reference mRNA molecule, and/or an ITGA1 reference cDNA molecule.

In any of the embodiments described throughout the present disclosure, the probes (such as, for example, an alteration-specific probe) can comprise a label. In some embodiments, the label is a fluorescent label, a radiolabel, or biotin.

The present disclosure also provides supports comprising a substrate to which any one or more of the probes disclosed herein is attached. Solid supports are solid-state substrates or supports with which molecules, such as any of the probes disclosed herein, can be associated. A form of solid support is an array. Another form of solid support is an array detector. An array detector is a solid support to which multiple different probes have been coupled in an array, grid, or other organized pattern. A form for a solid-state substrate is a microtiter dish, such as a standard 96-well type. In some embodiments, a multiwell glass slide can be employed that normally contains one array per well. In some embodiments, the support is a microarray.

In some embodiments, any of the methods described herein can further comprise determining the subject’s burden of having an ITGA1 variant nucleic acid molecule, and/or an ITGA1 predicted loss-of-function variant polypeptide associated with a decreased risk of developing a fibrotic lung disease, pulmonary fibrosis, interstitial lung disease, COPD, or asthma. The burden is the aggregate of all variants in the ITGA1 gene, which can be carried out in an association analysis with a lung disease, such as a fibrotic lung disease, pulmonary fibrosis, interstitial lung disease, COPD, or asthma. In some embodiments, the subject is homozygous for one or more ITGA1 variant nucleic acid molecules associated with a decreased risk of developing a fibrotic lung disease, pulmonary fibrosis, interstitial lung disease, COPD, or asthma. In some embodiments, the subject is heterozygous for one or more ITGA1 variant nucleic acid molecules associated with a decreased risk of developing a fibrotic lung disease, pulmonary fibrosis, interstitial lung disease, COPD, or asthma. The result of the association analysis suggests that ITGA1 variant nucleic acid molecules are associated with decreased risk of developing a fibrotic lung disease, pulmonary fibrosis, interstitial lung disease, COPD, or asthma. When the subject has a lower burden, the subject has an increased risk of developing a fibrotic lung disease, pulmonary fibrosis, interstitial lung disease, COPD, or asthma compared to subjects having a higher burden, and the subject is administered or continued to be administered the lung disease therapeutic agent in a standard dosage amount, and/or an ITGA1 inhibitor. When the subject has a higher burden, the subject has a decreased risk of developing a fibrotic lung disease, pulmonary fibrosis, interstitial lung disease, COPD, or asthma compared to subjects having a lower burden and the subject is administered or continued to be administered the lung disease therapeutic agent in an amount that is the same as or less than the standard dosage amount. The higher the burden, the lower the risk of developing a fibrotic lung disease, pulmonary fibrosis, interstitial lung disease, COPD, or asthma. The ITGA1 variant nucleic acid molecule can be any of the ITGA1 variant nucleic acid molecules having any of the variations described herein.

In some embodiments, the subject’s burden of having any one or more ITGA1 variant nucleic acid molecules represents a weighted aggregate of a plurality of ITGA1 variant nucleic acid molecules. In some embodiments, the burden is calculated using at least about 2, at least about 3, at least about 4, at least about 5, at least about 10, at least about 20, at least about 30, at least about 40, at least about 50, at least about 60, at least about 70, at least about 80, at least about 100, at least about 120, at least about 150, at least about 200, at least about 250, at least about 300, at least about 400, at least about 500, at least about 1,000, at least about 10,000, at least about 100,000, or at least about or more than 1,000,000 genetic variants present in or around (up to 10 Mb) the ITGA1 gene where the genetic burden is the number of alleles multiplied by the association estimate with fibrotic lung disease or related outcome for each allele (e.g., a weighted polygenic burden score). This can include any genetic variants, regardless of their genomic annotation, in proximity to the ITGA1 gene (up to 10 Mb around the gene) that show a non-zero association with fibrotic lung disease-related traits in a genetic association analysis. In some embodiments, when the subject has a burden higher than a desired threshold score, the subject has a decreased risk of developing a fibrotic lung disease, pulmonary fibrosis, interstitial lung disease, COPD, or asthma. In some embodiments, when the subject has a burden lower than a desired threshold score, the subject has an increased risk of developing a fibrotic lung disease, pulmonary fibrosis, interstitial lung disease, COPD, or asthma.

In some embodiments, the burden may be divided into quintiles, e.g., top quintile, intermediate quintile, and bottom quintile, wherein the top quintile of burden corresponds to the lowest risk group and the bottom quintile of burden corresponds to the highest risk group. In some embodiments, a subject having a greater burden comprises the highest weighted burdens, including, but not limited to the top 10%, top 20%, top 30%, top 40%, or top 50% of burdens from a subject population. In some embodiments, the genetic variants comprise the genetic variants having association with fibrotic lung disease in the top 10%, top 20%, top 30%, top 40%, or top 50% of p-value range for the association. In some embodiments, each of the identified genetic variants comprise the genetic variants having association with a fibrotic lung disease, pulmonary fibrosis, interstitial lung disease, COPD, or asthma with p-value of no more than about 10⁻², about 10⁻³, about 10⁻⁴, about 10⁻⁵, about 10⁻⁶, about 10⁻⁷, about 10⁻⁸, about 10⁻⁹, about 10⁻¹⁰, about 10⁻¹¹, about 10⁻¹², about 10⁻¹³, about 10⁻¹⁴, or about or 10⁻¹⁵. In some embodiments, the identified genetic variants comprise the genetic variants having association with a fibrotic lung disease, pulmonary fibrosis, interstitial lung disease, COPD, or asthma with p-value of less than 5×10⁻⁸. In some embodiments, the identified genetic variants comprise genetic variants having association with a fibrotic lung disease, pulmonary fibrosis, interstitial lung disease, COPD, or asthma in high-risk subjects as compared to the rest of the reference population with odds ratio (OR) about 1.5 or greater, about 1.75 or greater, about 2.0 or greater, or about 2.25 or greater for the top 20% of the distribution; or about 1.5 or greater, about 1.75 or greater, about 2.0 or greater, about 2.25 or greater, about 2.5 or greater, or about 2.75 or greater. In some embodiments, the odds ratio (OR) may range from about 1.0 to about 1.5, from about 1.5 to about 2.0, from about 2.0 to about 2.5, from about 2.5 to about 3.0, from about 3.0 to about 3.5, from about 3.5 to about 4.0, from about 4.0 to about 4.5, from about 4.5 to about 5.0, from about 5.0 to about 5.5, from about 5.5 to about 6.0, from about 6.0 to about 6.5, from about 6.5 to about 7.0, or greater than 7.0. In some embodiments, high-risk subjects comprise subjects having burdens in the bottom decile, quintile, or tertile in a reference population. The threshold of the burden is determined on the basis of the nature of the intended practical application and the risk difference that would be considered meaningful for that practical application.

In some embodiments, when a subject is identified as having an increased risk of developing a fibrotic lung disease, pulmonary fibrosis, interstitial lung disease, COPD, or asthma, the subject is further administered a lung disease therapeutic agent, and/or an ITGA1 inhibitor, as described herein. For example, when the subject is ITGA1 reference, and therefore has an increased risk of developing a fibrotic lung disease, pulmonary fibrosis, interstitial lung disease, COPD, or asthma, the subject is administered an ITGA1 inhibitor. In some embodiments, such a subject is also administered a lung disease therapeutic agent. In some embodiments, when the subject is heterozygous for an ITGA1 variant nucleic acid molecule, the subject is administered the lung disease therapeutic agent in a dosage amount that is the same as or less than a standard dosage amount, and is also administered an ITGA1 inhibitor. In some embodiments, the subject is ITGA1 reference. In some embodiments, the subject is heterozygous for an ITGA1 variant nucleic acid molecule. Furthermore, when the subject has a lower burden for having an ITGA1 variant nucleic acid molecule, and therefore has an increased risk of developing a fibrotic lung disease, pulmonary fibrosis, interstitial lung disease, COPD, or asthma, the subject is administered a lung disease therapeutic agent. In some embodiments, when the subject has a lower burden for having an ITGA1 variant nucleic acid molecule, the subject is administered the lung disease therapeutic agent in a dosage amount that is the same as or greater than the standard dosage amount administered to a subject who has a greater burden for having an ITGA1 variant nucleic acid molecule.

The nucleotide sequence of an ITGA1 reference genomic nucleic acid molecule is set forth in SEQ ID NO:1.

The nucleotide sequence of an ITGA1 reference mRNA molecule is set forth in SEQ ID NO:2. The nucleotide sequence of another ITGA1 reference mRNA molecule is set forth in SEQ ID NO:2. The nucleotide sequence of another ITGA1 reference mRNA molecule is set forth in SEQ ID NO:3. The nucleotide sequence of another ITGA1 reference mRNA molecule is set forth in SEQ ID NO:5.

The nucleotide sequence of an ITGA1 reference cDNA molecule is set forth in SEQ ID NO:6. The nucleotide sequence of another ITGA1 reference cDNA molecule is set forth in SEQ ID NO:7. The nucleotide sequence of another ITGA1 reference cDNA molecule is set forth in SEQ ID NO:8. The nucleotide sequence of another ITGA1 reference cDNA molecule is set forth in SEQ ID NO:9.

The genomic nucleic acid molecules, mRNA molecules, and cDNA molecules can be from any organism. For example, the genomic nucleic acid molecules, mRNA molecules, and cDNA molecules can be human or an ortholog from another organism, such as a non-human mammal, a rodent, a mouse, or a rat. It is understood that gene sequences within a population can vary due to polymorphisms such as single-nucleotide polymorphisms. The examples provided herein are only exemplary sequences. Other sequences are also possible.

Also provided herein are functional polynucleotides that can interact with the disclosed nucleic acid molecules. Examples of functional polynucleotides include, but are not limited to, antisense molecules, aptamers, ribozymes, triplex forming molecules, and external guide sequences. The functional polynucleotides can act as effectors, inhibitors, modulators, and stimulators of a specific activity possessed by a target molecule, or the functional polynucleotides can possess a de novo activity independent of any other molecules.

The isolated nucleic acid molecules, or the complement thereof, can also be present within a host cell. In some embodiments, the host cell can comprise the vector that comprises any of the nucleic acid molecules described herein, or the complement thereof. In some embodiments, the nucleic acid molecule is operably linked to a promoter active in the host cell. In some embodiments, the promoter is an exogenous promoter. In some embodiments, the promoter is an inducible promoter. In some embodiments, the host cell is a bacterial cell, a yeast cell, an insect cell, or a mammalian cell. In some embodiments, the host cell is a bacterial cell. In some embodiments, the host cell is a yeast cell. In some embodiments, the host cell is an insect cell. In some embodiments, the host cell is a mammalian cell.

Desired regulatory sequences for mammalian host cell expression can include, for example, viral elements that direct high levels of polypeptide expression in mammalian cells, such as promoters and/or enhancers derived from retroviral LTRs, cytomegalovirus (CMV) (such as, for example, CMV promoter/enhancer), Simian Virus 40 (SV40) (such as, for example, SV40 promoter/enhancer), adenovirus, (such as, for example, the adenovirus major late promoter (AdMLP)), polyoma and strong mammalian promoters such as native immunoglobulin and actin promoters. Methods of expressing polypeptides in bacterial cells or fungal cells (such as, for example, yeast cells) are also well known. A promoter can be, for example, a constitutively active promoter, a conditional promoter, an inducible promoter, a temporally restricted promoter (such as, for example, a developmentally regulated promoter), or a spatially restricted promoter (such as, for example, a cell-specific or tissue-specific promoter).

Percent identity (or percent complementarity) between particular stretches of nucleotide sequences within nucleic acid molecules or amino acid sequences within polypeptides can be determined routinely using BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482-489). Herein, if reference is made to percent sequence identity, the higher percentages of sequence identity are preferred over the lower ones.

As used herein, the phrase “corresponding to” or grammatical variations thereof when used in the context of the numbering of a particular nucleotide or nucleotide sequence or position refers to the numbering of a specified reference sequence when the particular nucleotide or nucleotide sequence is compared to a reference sequence (such as, for example, SEQ ID NO:1, SEQ ID NO:2, or SEQ ID NO:6). In other words, the residue (such as, for example, nucleotide or amino acid) number or residue (such as, for example, nucleotide or amino acid) position of a particular polymer is designated with respect to the reference sequence rather than by the actual numerical position of the residue within the particular nucleotide or nucleotide sequence. For example, a particular nucleotide sequence can be aligned to a reference sequence by introducing gaps to optimize residue matches between the two sequences. In these cases, although the gaps are present, the numbering of the residue in the particular nucleotide or nucleotide sequence is made with respect to the reference sequence to which it has been aligned.

As described herein, a position within an ITGA1 variant genomic nucleic acid molecule that corresponds to a variant position, for example, can be identified by performing a sequence alignment between the nucleotide sequence of a particular ITGA1 nucleic acid molecule and the nucleotide sequence of the variant nucleic acid molecule. A variety of computational algorithms exist that can be used for performing a sequence alignment to identify a nucleotide position that corresponds to, for example, the variant position of an ITGA1 variant nucleic acid molecule. For example, by using the NCBI BLAST algorithm (Altschul et al., Nucleic Acids Res., 1997, 25, 3389-3402) or CLUSTALW software (Sievers and Higgins, Methods Mol. Biol., 2014, 1079, 105-116) sequence alignments may be performed. However, sequences can also be aligned manually.

The amino acid sequences of ITGA1 reference polypeptides are set forth in SEQ ID NO:10 (Isoform 1) and SEQ ID NO:11 (Isoform 2). Referring to SEQ ID NO:10 (Isoform 1), the ITGA1 reference polypeptide is 1,179 amino acids in length. Referring to SEQ ID NO:11 (Isoform 2), the ITGA1 reference polypeptide is 1,173 amino acids in length.

The nucleotide and amino acid sequences listed in the accompanying sequence listing are shown using standard letter abbreviations for nucleotide bases, and three-letter code for amino acids. The nucleotide sequences follow the standard convention of beginning at the 5′ end of the sequence and proceeding forward (i.e., from left to right in each line) to the 3′ end. Only one strand of each nucleotide sequence is shown, but the complementary strand is understood to be included by any reference to the displayed strand. The amino acid sequence follows the standard convention of beginning at the amino terminus of the sequence and proceeding forward (i.e., from left to right in each line) to the carboxy terminus.

The present disclosure also provides lung disease therapeutic agents for use in the treatment of a fibrotic lung disease, pulmonary fibrosis, interstitial lung disease, COPD, or asthma (or for use in the preparation of a medicament for treating a fibrotic lung disease, pulmonary fibrosis, interstitial lung disease, COPD, or asthma) in a subject, wherein the subject has any of the ITGA1 variant genomic nucleic acid molecules, variant mRNA molecules, and/or variant cDNA molecules described herein. The lung disease therapeutic agents can be any of the lung disease therapeutic agents described herein.

In some embodiments, the subject is identified as having an ITGA1 variant genomic nucleic acid molecule, wherein the genomic nucleic acid molecule has a nucleotide sequence comprising a nucleotide at a position corresponding to the nucleotide at the variant position of an ITGA1 variant nucleic acid molecule, or the complement thereof.

In some embodiments, the subject is identified as having an ITGA1 variant mRNA molecule, wherein the mRNA molecule has a nucleotide sequence comprising a nucleotide at a position corresponding to the nucleotide at the variant position of an ITGA1 variant nucleic acid molecule, or the complement thereof.

In some embodiments, the subject is identified as having an ITGA1 variant cDNA molecule, wherein the cDNA molecule has a nucleotide sequence comprising a nucleotide at a position corresponding to the nucleotide at the variant position of an ITGA1 variant nucleic acid molecule, or the complement thereof.

In some embodiments, the subject is identified as having: i) an ITGA1 variant genomic nucleic acid molecule having a nucleotide sequence comprising a nucleotide at a position corresponding to the nucleotide at the variant position of an ITGA1 variant nucleic acid molecule, or the complement thereof; ii) an mRNA molecule having a nucleotide sequence comprising a nucleotide at a position corresponding to the nucleotide at the variant position of an ITGA1 variant nucleic acid molecule, or the complement thereof; or iii) a cDNA molecule having a nucleotide sequence comprising a nucleotide at a position corresponding to the nucleotide at the variant position of an ITGA1 variant nucleic acid molecule, or the complement thereof.

In some embodiments, the subject is identified as having an ITGA1 variant genomic nucleic acid molecule having a nucleotide sequence comprising a nucleotide at a position corresponding to the nucleotide at the variant position of an ITGA1 variant nucleic acid molecule, or the complement thereof.

In some embodiments, the subject is identified as having an ITGA1 variant mRNA molecule having a nucleotide sequence comprising a nucleotide at a position corresponding to the nucleotide at the variant position of an ITGA1 variant nucleic acid molecule, or the complement thereof.

In some embodiments, the subject is identified as having an ITGA1 variant cDNA molecule having a nucleotide sequence comprising a nucleotide at a position corresponding to the nucleotide at the variant position of an ITGA1 variant nucleic acid molecule, or the complement thereof.

In some embodiments, the subject is identified as having an ITGA1 predicted loss-of-function polypeptide that comprises an amino acid sequence comprising a variation encoded by any of the ITGA1 variant nucleic acid molecules described herein.

The present disclosure also provides ITGA1 inhibitors for use in the treatment of a fibrotic lung disease, pulmonary fibrosis, interstitial lung disease, COPD, or asthma (or for use in the preparation of a medicament for treating a fibrotic lung disease, pulmonary fibrosis, interstitial lung disease, COPD, or asthma) in a subject, wherein the subject is heterozygous for any of the ITGA1 variant genomic nucleic acid molecules, variant mRNA molecules, and/or variant cDNA molecules described herein, or wherein the subject is reference for an ITGA1 genomic nucleic acid molecule, mRNA molecule, or cDNA molecule. The ITGA1 inhibitors can be any of the ITGA1 inhibitors described herein.

In some embodiments, the subject is reference for an ITGA1 genomic nucleic acid molecule, an ITGA1 mRNA molecule, or an ITGA1 cDNA molecule. In some embodiments, the subject is reference for an ITGA1 genomic nucleic acid molecule. In some embodiments, the subject is reference for an ITGA1 mRNA molecule. In some embodiments, the subject is reference for an ITGA1 cDNA molecule.

In some embodiments, the subject is identified as being heterozygous for an ITGA1 variant genomic nucleic acid molecule, wherein the genomic nucleic acid molecule has a nucleotide sequence comprising a nucleotide at a position corresponding to the nucleotide at the variant position of an ITGA1 variant nucleic acid molecule, or the complement thereof.

In some embodiments, the subject is identified as being heterozygous for an ITGA1 variant mRNA molecule, wherein the mRNA molecule has a nucleotide sequence comprising a nucleotide at a position corresponding to the nucleotide at the variant position of an ITGA1 variant nucleic acid molecule, or the complement thereof.

In some embodiments, the subject is identified as being heterozygous for an ITGA1 variant cDNA molecule, wherein the cDNA molecule has a nucleotide sequence comprising a nucleotide at a position corresponding to the nucleotide at the variant position of an ITGA1 variant nucleic acid molecule, or the complement thereof.

In some embodiments, the subject is identified as being heterozygous for: i) an ITGA1 variant genomic nucleic acid molecule having a nucleotide sequence comprising a nucleotide at a position corresponding to the nucleotide at the variant position of an ITGA1 variant nucleic acid molecule, or the complement thereof; ii) an ITGA1 variant mRNA molecule having a nucleotide sequence comprising a nucleotide at a position corresponding to the nucleotide at the variant position of an ITGA1 variant nucleic acid molecule, or the complement thereof; or iii) an ITGA1 variant cDNA molecule having a nucleotide sequence comprising a nucleotide at a position corresponding to the nucleotide at the variant position of an ITGA1 variant nucleic acid molecule, or the complement thereof.

In some embodiments, the subject is identified as being heterozygous for an ITGA1 variant genomic nucleic acid molecule having a nucleotide sequence comprising a nucleotide at a position corresponding to the nucleotide at the variant position of an ITGA1 variant nucleic acid molecule, or the complement thereof.

In some embodiments, the subject is identified as being heterozygous for an ITGA1 variant mRNA molecule having a nucleotide sequence comprising a nucleotide at a position corresponding to the nucleotide at the variant position of an ITGA1 variant nucleic acid molecule, or the complement thereof.

In some embodiments, the subject is identified as being heterozygous for an ITGA1 variant cDNA molecule having a nucleotide sequence comprising a nucleotide at a position corresponding to the nucleotide at the variant position of an ITGA1 variant nucleic acid molecule, or the complement thereof.

All patent documents, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable. Likewise, if different versions of a publication, website or the like are published at different times, the version most recently published at the effective filing date of the application is meant unless otherwise indicated. Any feature, step, element, embodiment, or aspect of the present disclosure can be used in combination with any other feature, step, element, embodiment, or aspect unless specifically indicated otherwise. Although the present disclosure has been described in some detail by way of illustration and example for purposes of clarity and understanding, it will be apparent that certain changes and modifications may be practiced within the scope of the appended claims.

The following examples are provided to describe the embodiments in greater detail. They are intended to illustrate, not to limit, the claimed embodiments. The following examples provide those of ordinary skill in the art with a disclosure and description of how the compounds, compositions, articles, devices and/or methods described herein are made and evaluated, and are intended to be purely exemplary and are not intended to limit the scope of any claims. Efforts have been made to ensure accuracy with respect to numbers (such as, for example, amounts, temperature, etc.), but some errors and deviations may be accounted for. Unless indicated otherwise, parts are parts by weight, temperature is in °C or is at ambient temperature, and pressure is at or near atmospheric.

### EXAMPLES

### Example 1: Association Between Loss-of-Function Variants in ITGA1 and Improved Lung Function

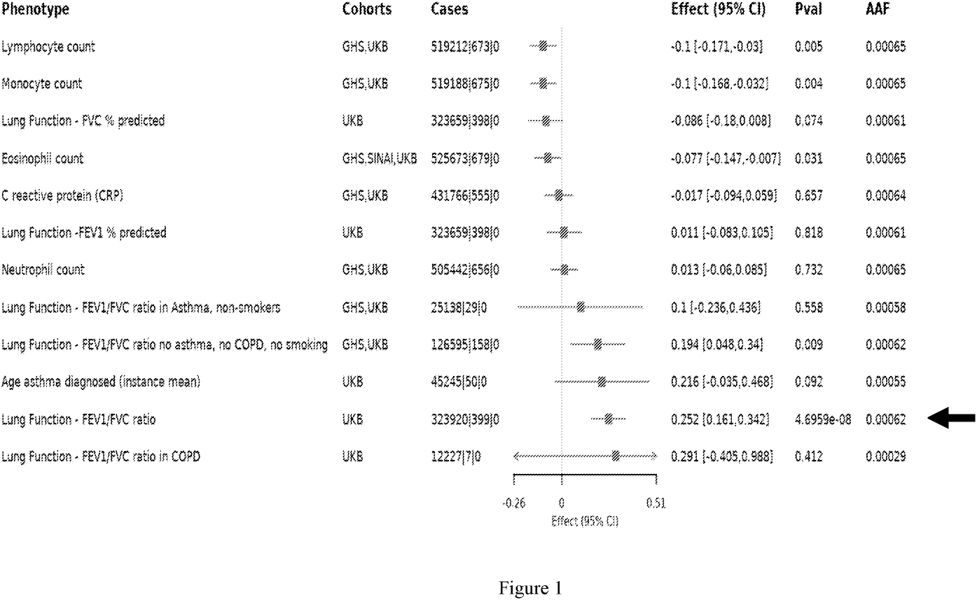

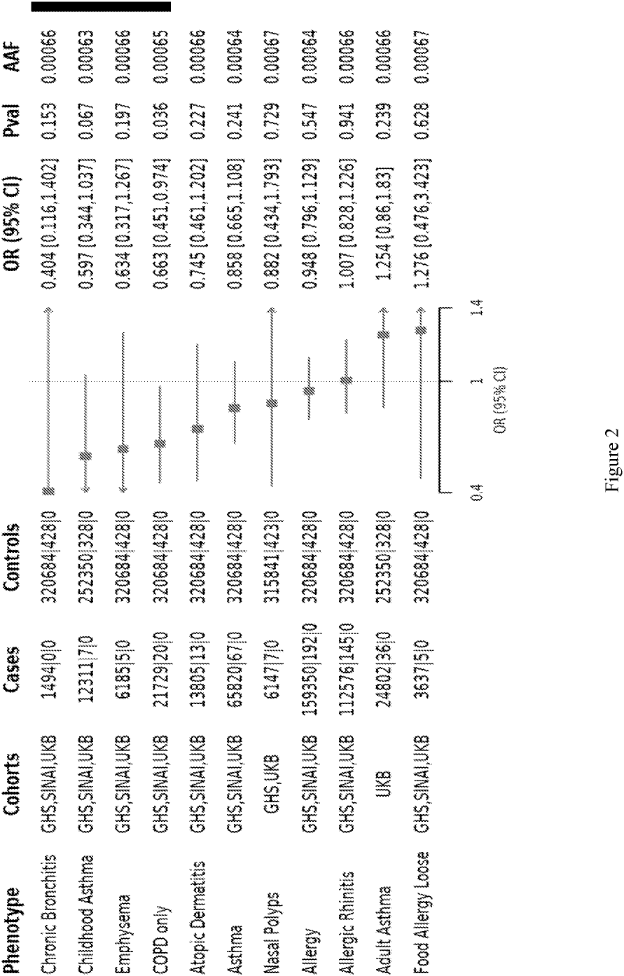

The association between common and rare variants and three lung function traits was examined: forced expiratory volume in 1 second (FEV1), forced vital capacity (FVC), and the ratio FEV1/FVC, which is a measurement of airway obstruction. An association between rare (minor allele frequency <1%) predicted loss-of-function variants in ITGA1 and improved lung function (greater FEV1/FVC ratio; FIG. 1) was identified. Nominal associations trending toward protection from asthma and COPD was also observed, suggesting loss of ITGA1 improves lung function and may be protective in asthma/COPD or other fibrotic lung diseases (FIG. 2).

Various modifications of the described subject matter, in addition to those described herein, will be apparent to those skilled in the art from the foregoing description. Such modifications are also intended to fall within the scope of the appended claims. Each reference (including, but not limited to, journal articles, U.S. and non-U.S. patents, patent application publications, international patent application publications, gene bank accession numbers, and the like) cited in the present application is incorporated herein by reference in its entirety and for all purposes.

1. A method of treating a subject having a fibrotic lung disease or at risk of developing a fibrotic lung disease, having pulmonary fibrosis or at risk of developing pulmonary fibrosis, having interstitial lung disease or at risk of developing interstitial lung disease, having chronic obstructive pulmonary disease (COPD) or at risk of developing COPD, and/or having asthma or at risk of developing asthma, the method comprising administering an Integrin Subunit Alpha 1 (ITGA1) inhibitor to the subject.

2-5. (canceled)

6. The method according to claim 1, wherein the ITGA1 inhibitor comprises an inhibitory nucleic acid molecule.

7. The method according to claim 6, wherein the inhibitory nucleic acid molecule comprises an antisense nucleic acid molecule, a small interfering RNA (siRNA), or a short hairpin RNA (shRNA) that hybridizes to an ITGA1 nucleic acid molecule.

8-14. (canceled)

15. The method according to claim 1, further comprising detecting the presence or absence of an ITGA1 variant nucleic acid molecule in a biological sample obtained from the subject.

16. The method according to claim 15, further comprising administering a lung disease therapeutic agent in a standard dosage amount to a subject wherein the ITGA1 variant nucleic acid molecule is absent from the biological sample.

17. The method according to claim 15, further comprising administering a lung disease therapeutic agent in a dosage amount that is the same as or less than a standard dosage amount to a subject that is heterozygous for the ITGA1 variant nucleic acid molecule.

18. The method according to claim 15, wherein the ITGA1 variant nucleic acid molecule is chosen from 5:52920430:C:T, 5:52944942:G:T, 5:52910243:G:T, 5:52918899:G:C, 5:52918730:A:T, 5:52952404:CA:C, 5:52861561:T:C, 5:52939693:T:C, 5:52910201:C:T, 5:52788416:T:C, 5:52788356:G:A, 5:52893783:G:T, 5:52920332:AT:A, 5:52952436:GA:G, 5:52939945:G:T, 5:52925371:C:T, 5:52939690:TG:T, 5:52898385:T:C, 5:52897529:GT:G, 5:52937490:C:G, 5:52947412:CT:C, 5:52882022:G:T, 5:52939946:T:A, 5:52861503:A:AT, 5:52881929:T:G, 5:52932134:C:A, 5:52939933:AC:A, 5:52933993:C:G, 5:52927645:TCCTG:T, 5:52939934:CT:C, 5:52893841:G:C, 5:52881939:G:T, 5:52932134:C:G, 5:52864814:GA:G, 5:52849381:C:A, 5:52922818:CT:C, 5:52925286:TG:T, 5:52865083:G:A, 5:52939606:C:CAT, 5:52915509:C:CGTGGTGA, 5:52865083:G:T, 5:52944999:ATC:A, 5:52910194:TA:T, 5:52947462:G:A, 5:52898323:C:T, 5:52945006:GT:G, 5:52910292:CT:C, 5:52861560:G:A, 5:52939642:G:GA, 5:52864979:GC:G, 5:52927590:CA:C, 5:52918814:G:T, 5:52788351:G:GC, 5:52920469:G:A, 5:52905808:A:AT, 5:52922876:GT:G, 5:52861501:C:CT, 5:52865771:T:A, 5:52947456:TGG:T, 5:52918736:C:T, 5:52920376:C:T, 5:52915496:G:GA, 5:52898346:GTC:G, 5:52947462:G:T, 5:52920444:C:A, 5:52882022:G:A, 5:52927640:TC:T, 5:52925470:G:GT, 5:52952430:GA:G, 5:52937516:T:C, 5:52915570:G:GC, 5:52861530:C:A, 5:52932046:G:A, 5:52893841:G:T, 5:52887965:TGTAA:T, 5:52937399:AG:A, 5:52887863:AG:A, 5:52905910:T:A, 5:52925304:TA:T, 5:52910222:C:T, 5:52887906:C:T, 5:52910199:AG:A, 5:52909042:G:A, 5:52898269:TA:T, 5:52939588:A:C, 5:52937515:G:GT, 5:52865745:C:A, 5:52937474:GT:G, 5:52925299:G:T, 5:52920367:CA:C, 5:52898296:CAG:C, 5:52925283:CT:C, 5:52893817:GAGAA:G, 5:52915565:TG:T, 5:52933892:A:T, 5:52915524:G:T, 5:52887846:C:T, 5:52937515:G:A, 5:52929670:GA:G, 5:52915463:G:A, 5:52905900:G:T, 5:52865688:AG:A, 5:52933993:C:A, 5:52932137:G:A, 5:52788355:T:C, 5:52897529:G:A, 5:52947344:G:A, 5:52910417:CAAGT:C, 5:52864772:C:A, 5:52939677:C:T, 5:52918807:C:A, 5:52915502:TTTTGG:T, 5:52893693:C:T, 5:52939855:A:T, 5:52925489:T:C, 5:52939673:C:CA, 5:52887966:G:T, 5:52918877:GA:G, and 5:52947463:T:C.

19. The method according to claim 15, wherein the detecting step is carried out in vitro.

20. The method according to claim 15, wherein the detecting step comprises sequencing at least a portion of the nucleotide sequence of the ITGA1 genomic nucleic acid molecule, or the complement thereof, in the biological sample, wherein the sequenced portion comprises a position corresponding to the variant position of an ITGA1 variant nucleic acid molecule, or the complement thereof;
wherein when the sequenced portion of the ITGA1 genomic nucleic acid molecule in the biological sample comprises a nucleotide sequence that comprises a nucleotide at a position corresponding to the nucleotide at the variant position of an ITGA1 variant nucleic acid molecule, then the ITGA1 genomic nucleic acid molecule in the biological sample is an ITGA1 variant genomic nucleic acid molecule.

21. The method according to claim 15, wherein the detecting step comprises sequencing at least a portion of the nucleotide sequence of the ITGA1 mRNA molecule in the biological sample, wherein the sequenced portion comprises a position corresponding to the variant position of an ITGA1 variant nucleic acid molecule, or the complement thereof;
wherein when the sequenced portion of the ITGA1 mRNA molecule in the biological sample comprises a nucleotide sequence that comprises a nucleotide at a position corresponding to the nucleotide at the variant position of an ITGA1 variant nucleic acid molecule, then the ITGA1 mRNA molecule in the biological sample is an ITGA1 variant mRNA molecule.

22-33. (canceled)

34. A method of treating a subject with a lung disease therapeutic agent, wherein the subject has a fibrotic lung disease, pulmonary fibrosis, interstitial lung disease, chronic obstructive pulmonary disease (COPD), or asthma, or wherein the subject is at risk of developing a fibrotic lung disease, pulmonary fibrosis, interstitial lung disease, COPD, or asthma, the method comprising:
determining whether the subject has an Integrin Subunit Alpha 1 (ITGA1) variant nucleic acid molecule by:
obtaining or having obtained a biological sample from the subject; and
performing or having performed a sequence analysis on the biological sample to determine if the subject has a genotype comprising the ITGA1 variant nucleic acid molecule; and
administering or continuing to administer the lung disease therapeutic agent in a standard dosage amount to a subject that is ITGA1 reference, and/or administering an ITGA1 inhibitor to the subject; and
administering or continuing to administer the lung disease therapeutic agent in an amount that is the same as or less than a standard dosage amount to a subject that is heterozygous for the ITGA1 variant nucleic acid molecule, and/or administering an ITGA1 inhibitor to the subj ect;
wherein the presence of a genotype having the ITGA1 variant nucleic acid molecule indicates the subject has a reduced risk of developing the fibrotic lung disease, pulmonary fibrosis, interstitial lung disease, COPD, or asthma.

35. The method according to claim 34, wherein the subject is ITGA1 reference, and the subject is administered or continued to be administered the lung disease therapeutic agent in a standard dosage amount, and is administered an ITGA1 inhibitor.

36. The method according to claim 34, wherein the subject is heterozygous for an ITGA1 variant nucleic acid molecule, and the subject is administered or continued to be administered the lung disease therapeutic agent in an amount that is the same as or less than a standard dosage amount, and is administered an ITGA1 inhibitor.

37. The method according to claim 34, wherein the ITGA1 variant nucleic acid molecule is chosen from 5:52920430:C:T, 5:52944942:G:T, 5:52910243:G:T, 5:52918899:G:C, 5:52918730:A:T, 5:52952404:CA:C, 5:52861561:T:C, 5:52939693:T:C, 5:52910201:C:T, 5:52788416:T:C, 5:52788356:G:A, 5:52893783:G:T, 5:52920332:AT:A, 5:52952436:GA:G, 5:52939945:G:T, 5:52925371:C:T, 5:52939690:TG:T, 5:52898385:T:C, 5:52897529:GT:G, 5:52937490:C:G, 5:52947412:CT:C, 5:52882022:G:T, 5:52939946:T:A, 5:52861503:A:AT, 5:52881929:T:G, 5:52932134:C:A, 5:52939933:AC:A, 5:52933993:C:G, 5:52927645:TCCTG:T, 5:52939934:CT:C, 5:52893841:G:C, 5:52881939:G:T, 5:52932134:C:G, 5:52864814:GA:G, 5:52849381:C:A, 5:52922818:CT:C, 5:52925286:TG:T, 5:52865083:G:A, 5:52939606:C:CAT, 5:52915509:C:CGTGGTGA, 5:52865083:G:T, 5:52944999:ATC:A, 5:52910194:TA:T, 5:52947462:G:A, 5:52898323:C:T, 5:52945006:GT:G, 5:52910292:CT:C, 5:52861560:G:A, 5:52939642:G:GA, 5:52864979:GC:G, 5:52927590:CA:C, 5:52918814:G:T, 5:52788351:G:GC, 5:52920469:G:A, 5:52905808:A:AT, 5:52922876:GT:G, 5:52861501:C:CT, 5:52865771:T:A, 5:52947456:TGG:T, 5:52918736:C:T, 5:52920376:C:T, 5:52915496:G:GA, 5:52898346:GTC:G, 5:52947462:G:T, 5:52920444:C:A, 5:52882022:G:A, 5:52927640:TC:T, 5:52925470:G:GT, 5:52952430:GA:G, 5:52937516:T:C, 5:52915570:G:GC, 5:52861530:C:A, 5:52932046:G:A, 5:52893841:G:T, 5:52887965:TGTAA:T, 5:52937399:AG:A, 5:52887863:AG:A, 5:52905910:T:A, 5:52925304:TA:T, 5:52910222:C:T, 5:52887906:C:T, 5:52910199:AG:A, 5:52909042:G:A, 5:52898269:TA:T, 5:52939588:A:C, 5:52937515:G:GT, 5:52865745:C:A, 5:52937474:GT:G, 5:52925299:G:T, 5:52920367:CA:C, 5:52898296:CAG:C, 5:52925283:CT:C, 5:52893817:GAGAA:G, 5:52915565:TG:T, 5:52933892:A:T, 5:52915524:G:T, 5:52887846:C:T, 5:52937515:G:A, 5:52929670:GA:G, 5:52915463:G:A, 5:52905900:G:T, 5:52865688:AG:A, 5:52933993:C:A, 5:52932137:G:A, 5:52788355:T:C, 5:52897529:G:A, 5:52947344:G:A, 5:52910417:CAAGT:C, 5:52864772:C:A, 5:52939677:C:T, 5:52918807:C:A, 5:52915502:TTTTGG:T, 5:52893693:C:T, 5:52939855:A:T, 5:52925489:T:C, 5:52939673:C:CA, 5:52887966:G:T, 5:52918877:GA:G, and 5:52947463:T:C.

38. The method according to claim 34, wherein the sequence analysis comprises sequencing at least a portion of the nucleotide sequence of the ITGA1 genomic nucleic acid molecule, or the complement thereof, in the biological sample, wherein the sequenced portion comprises a position corresponding to the variant position of an ITGA1 variant nucleic acid molecule, or the complement thereof;
wherein when the sequenced portion of the ITGA1 genomic nucleic acid molecule, or the complement thereof, in the biological sample comprises a nucleotide sequence that comprises a nucleotide at a position corresponding to the nucleotide at the variant position of an ITGA1 variant nucleic acid molecule, then the ITGA1 genomic nucleic acid molecule in the biological sample is an ITGA1 variant genomic nucleic acid molecule.

39. The method according to claim 34, wherein the sequence analysis comprises sequencing at least a portion of the nucleotide sequence of the ITGA1 mRNA molecule, or the complement thereof, in the biological sample, wherein the sequenced portion comprises a position corresponding to the variant position of an ITGA1 variant nucleic acid molecule, or the complement thereof;
wherein when the sequenced portion of the ITGA1 mRNA molecule in the biological sample comprises a nucleotide sequence that comprises a nucleotide at a position corresponding to the nucleotide at the variant position of an ITGA1 variant nucleic acid molecule, then the ITGA1 mRNA molecule in the biological sample is an ITGA1 variant mRNA molecule.

40. The method according to claim 34, wherein the sequence analysis comprises sequencing at least a portion of the nucleotide sequence of the ITGA1 cDNA molecule, or the complement thereof, in the biological sample, wherein the sequenced portion comprises a position corresponding to the variant position of an ITGA1 variant nucleic acid molecule, or the complement thereof;
wherein when the sequenced portion of the ITGA1 cDNA molecule in the biological sample comprises a nucleotide sequence that comprises a nucleotide at a position corresponding to the nucleotide at the variant position of an ITGA1 variant nucleic acid molecule, then the ITGA1 cDNA molecule in the biological sample is an ITGA1 variant cDNA molecule.

41. The method according to claim 34, wherein the sequence analysis comprises:
a) contacting the biological sample with a primer hybridizing to a portion of the nucleotide sequence of the ITGA1 genomic nucleic acid molecule, or the complement thereof, that is proximate to a position corresponding to a variant position of an ITGA1 variant nucleic acid molecule;
b) extending the primer at least through the position of the nucleotide sequence of the ITGA1 genomic nucleic acid molecule, or the complement thereof, corresponding to the variant position of an ITGA1 variant nucleic acid molecule; and
c) determining whether the extension product of the primer comprises a nucleotide at a position corresponding to the nucleotide at the variant position of an ITGA1 variant nucleic acid molecule.

42-52. (canceled)

53. The method according to claim 34, wherein the ITGA1 inhibitor comprises an inhibitory nucleic acid molecule.

54. The method according to claim 53, wherein the inhibitory nucleic acid molecule comprises an antisense nucleic acid molecule, a small interfering RNA (siRNA), or a short hairpin RNA (shRNA) that hybridizes to an ITGA1 nucleic acid molecule.

55-91. (canceled)
